(19) United States
(12) Patent Application Publication
(10) Pub. No.: US 2025/0257391 A1
(43) Pub. Date: Aug. 14, 2025

(54) ROLLING CIRCLE AMPLIFICATION COMPRISING CROSSLINKING AND DE-CROSSLINKING

(71) Applicant: 10x Genomics, Inc., Pleasanton, CA (US)

(72) Inventor: Marco MIGNARDI, Sundbyberg (SE)

(21) Appl. No.: 19/053,140

(22) Filed: Feb. 13, 2025

Related U.S. Application Data
(60) Provisional application No. 63/553,573, filed on Feb. 14, 2024.

(51) Int. Cl. C12Q 1/6853

(52) U.S. Cl. CPC C12Q 1/6853

(57) ABSTRACT

The present disclosure in some aspects relates to methods, compositions, and kits for rolling circle amplification (RCA) comprising extending a nucleic acid priming sequence hybridized to a circular nucleic acid template using a polymerase to generate an extended priming sequence, wherein the circular nucleic acid template is crosslinked to a second nucleic acid strand; de-crosslinking the circular nucleic acid template from the second nucleic acid strand; and extending the extended priming sequence to generate a rolling circle amplification product (RCP).

### CROSS-REFERENCE TO RELATED APPLICATIONS

This application claims priority to U.S. Provisional Patent Application No. 63/553,573 filed Feb. 14, 2024, entitled “ROLLING CIRCLE AMPLIFICATION COMPRISING CROSSLINKING AND DE-CROSSLINKING,” the content of which is incorporated herein by reference in its entirety for all purposes.

### FIELD

The present disclosure generally relates in some aspects to methods and compositions for rolling circle amplification of nucleic acids.

### BACKGROUND

Genomic, transcriptomic, and proteomic profiling of cells and tissue samples using microscopic imaging can resolve multiple analytes of interest at the same time, thereby providing valuable information regarding analyte abundance and localization in situ. Thus, these in situ assays are important tools, for example, for understanding the molecular basis of cell identity and developing treatment for diseases. In multiplex assays where multiple signals are detected simultaneously, it is important that as much information as possible is collected. However, due to the heterogeneity of analyte abundance (e.g., gene expression levels) and variations among reactions at different locations of a sample, there can be a wide and heterogeneous size and intensity distribution of signals in the sample. Large signal spots may overlap with one another and/or mask adjacent smaller signal spots, rendering the smaller spots unresolvable. There is a need for new and improved methods for in situ assays. The present disclosure addresses these and other needs.

### SUMMARY

In some aspects, provided herein are methods, compositions, and kits that address issues such as lack of uniformity in the size of rolling circle amplification products. In certain embodiments, provided herein are methods, compositions, and kits useful for rolling circle amplification of a circular nucleic acid template. Before or after circularization, the circular nucleic acid template is crosslinked to a second nucleic acid strand. In certain cases, the second nucleic acid strand contains a priming sequence which is extended by a polymerase. In some embodiments, the second nucleic acid strand containing the priming sequence is a target nucleic acid molecule in a biological sample, or is an exogenous primer contacted with the biological sample. In other cases, the second nucleic acid strand crosslinked to the template does not contain the priming sequence extended by a polymerase. Advantageously, in some embodiments, a crosslink between the circular nucleic acid template and the second nucleic acid strand improves uniformity in RCA products by stalling the polymerase when it reaches the crosslink. In some aspects, the method further comprises de-crosslinking the circular nucleic acid template from the second nucleic acid strand, and continuing rolling circle amplification.

In some embodiments, provided herein are methods comprising (a) extending a nucleic acid priming sequence hybridized to a circular nucleic acid template using a polymerase to generate an extended priming sequence, wherein a first nucleic acid strand comprises the circular nucleic acid template, and the circular nucleic acid template is crosslinked to a second nucleic acid strand; (b) de-crosslinking the circular nucleic acid template from the second nucleic acid strand; and (c) extending the extended priming sequence to generate a rolling circle amplification product (RCP). In some embodiments, the second nucleic acid strand and the nucleic acid priming sequence are in two separate molecules.

In some embodiments, provided herein are methods comprising (a) extending a target nucleic acid hybridized to a circular nucleic acid template using a polymerase to generate an extended sequence, wherein a first nucleic acid strand comprises the circular nucleic acid template, and the circular nucleic acid template is crosslinked to a second nucleic acid strand separate from the target nucleic acid; (b) de-crosslinking the circular nucleic acid template from the second nucleic acid strand; and (c) extending the extended sequence to generate a rolling circle amplification product (RCP).

In some embodiments, the circular nucleic acid template is generated by ligating a padlock probe prior to (a).

In some embodiments, the method comprises providing a second polymerase after de-crosslinking the circular nucleic acid template from the second nucleic acid strand. In some embodiments, the second polymerase is the same type of polymerase used to extend the nucleic acid priming sequence in a previous extension (e.g., of the nucleic acid priming sequence hybridized to the circular nucleic acid or of the template target nucleic acid hybridized to the circular nucleic acid template.

In some embodiments, the polymerase is a Phi29 DNA polymerase or a Bst DNA polymerase. In some embodiments, the second polymerase is a Phi29 DNA polymerase or a Bst DNA polymerase.

In some embodiments, the method is performed in a biological sample, wherein the method comprises contacting the biological sample with the second nucleic acid strand and (i) contacting the biological sample with the circular nucleic acid template, wherein the circular nucleic acid template hybridizes to a target nucleic acid in the biological sample, or (ii) contacting the biological sample with a circularizable probe or probe set that hybridizes to a target nucleic acid in the biological sample, and ligating the circularizable probe or probe set to generate the circular nucleic acid template in the biological sample.

In some embodiments, the second nucleic acid strand is an oligonucleotide. In some embodiments, the method comprises contacting the biological sample with the second nucleic acid strand and the circular nucleic acid template or the circularizable probe or probe set simultaneously. In some embodiments, the method comprises contacting the biological sample with the second nucleic acid strand and the circular nucleic acid template or the circularizable probe or probe set sequentially.

In some embodiments, the second nucleic acid strand is crosslinked to the circular nucleic acid template or the circularizable probe or probe set before contacting the biological sample. In some embodiments, the method further comprises crosslinking the second nucleic acid strand to the circular nucleic acid template or the circularizable probe or probe set before contacting the biological sample. In some embodiments, the second nucleic acid strand is crosslinked to the circular nucleic acid template or the circularizable probe or probe set by irradiating a mixture comprising the second nucleic acid strand and the circular nucleic acid template or the circularizable probe or probe set with UV light prior to contacting the biological sample. In some embodiments, the mixture is irradiated using a 350-400 nm wavelength of light. In some embodiments, the wavelength is 360-370 nm.

In some embodiments, the mixture is irradiated for between about 1 second and about 1 minute. In some embodiments, the mixture is irradiated for between about 1 second and about 30 seconds.

In some embodiments, the method comprises crosslinking the second nucleic acid strand to the circular nucleic acid template or the circularizable probe or probe set after contacting the biological sample. In some embodiments, crosslinking the second nucleic acid strand to the circular nucleic acid template or the circularizable probe or probe set comprises irradiating the biological sample with UV light. In some embodiments, crosslinking the second nucleic acid strand to the circular nucleic acid template or the circularizable probe or probe set comprises irradiating the biological sample using a 350-400 nm wavelength of light. In some embodiments, the wavelength is 360-370 nm.

In some embodiments, the biological sample is irradiated for between about 1 second and about 1 minute. In some embodiments, the biological sample is irradiated for between about 1 second and about 30 seconds.

In some embodiments, the second nucleic acid strand comprises the nucleic acid priming sequence. In some embodiments, the second nucleic acid strand does not comprise the nucleic acid priming sequence. In some embodiments, the second nucleic acid strand is not capable of being extended by a polymerase. In some embodiments, the second nucleic acid strand comprises an irreversible terminating group. In some embodiments, the second nucleic acid strand comprises a 3′ dideoxynucleotide.

In some embodiments, the second nucleic acid strand is between about 5 and about 100 nucleotides in length.

In some embodiments, the method comprises contacting the circular nucleic acid with a primer oligonucleotide comprising the nucleic acid priming sequence. In some embodiments, the nucleic acid priming sequence is between about 5 and about 100 nucleotides in length.

In some embodiments, the target nucleic acid is RNA. In some embodiments, the target nucleic acid is cleaved to generate a free 3′ end of the target nucleic acid for extension. In some embodiments, the target nucleic acid is cleaved using an RNase H or a DNAzyme.

In some embodiments, the method comprises performing one or more stringent washes of the circular nucleic acid template or the circularizable probe or probe set crosslinked to the second nucleic acid strand.

In some embodiments, the circular nucleic acid template or the circularizable probe or probe set comprises a hybridization region that hybridizes to the target nucleic acid, and wherein the hybridization region comprises a crosslinkable moiety. In some embodiments, the circular nucleic acid template or the circularizable probe or probe set comprises a primer-binding region that hybridizes to the nucleic acid priming sequence, and wherein the primer-binding region comprises a nucleotide residue functionalized with a crosslinkable moiety. In some embodiments, the second nucleic acid strand comprises a crosslinkable moiety. In some embodiments, the crosslinkable moiety of the second nucleic acid strand is at the 5′ end of the second nucleic acid strand. In some embodiments, the crosslinkable moiety of the second nucleic acid strand is within 1, 2, 3, 4, or more nucleotides from the 5′ end of the second nucleic acid strand.

In some embodiments, provided herein is a method for analyzing a biological sample, comprising: (a) contacting the biological sample with a plurality of circularizable probes, wherein each circularizable probe comprises a hybridization region complementary to a different target nucleic acid sequence, and wherein at least a subset of the circularizable probes are crosslinked to primers before contacting the biological sample, (b) allowing the circularizable probes to hybridize to their respective target nucleic acid sequences present in the biological sample; (c) ligating the hybridized circularizable probes to generate a plurality of circular nucleic acid templates; (d) extending the primers using a polymerase to generate extended priming sequences using the circular nucleic acid templates as templates; (e) de-crosslinking the circular nucleic acid templates from the primers; and (f) extending the extended priming sequences to generate a plurality of rolling circle amplification products (RCPs) in the biological sample. In some embodiments, the method comprises performing one or more stringent washes of the biological sample after allowing the circularizable probes to hybridize to their respective target nucleic acid sequences present in the biological sample. In some embodiments, each of the circularizable probes is crosslinked to a primer before contacting the biological sample.

In some embodiments, the primers each comprise a crosslinkable moiety. In some embodiments, the crosslinkable moieties are crosslinked to the circularizable probe. In some embodiments, the crosslinkable moiety comprises a vinylcarbazone-based moiety In some embodiments, the crosslinkable moiety is a 3-cyanovinylcarbazole phosphoramidite or a pyranocarbazole phosphoramidite. In some embodiments, the crosslinkable moiety is a 3-cyanovinylcarbazole (^{CNV}K) nucleoside, a 3-cyanovinylcarbazole modified D-threoninol (^{CNV}D), a pyranocarbazole nucleoside (^{PC}X) or a pyranocarbazole modified D-threoninol (^{PCX}D). In some embodiments, the crosslinkable moiety comprises a coumarin.

In some embodiments, de-crosslinking the circular nucleic acid template from the second nucleic acid strand comprises irradiating the circular nucleic acid template crosslinked to the second nucleic acid strand. In some embodiments, the de-crosslinking comprises irradiating the sample with a wavelength between about 300 nm and about 320 nm. In some embodiments, the wavelength is between about 305 nm and about 312 nm. In some embodiments, the de-crosslinking comprises irradiating the sample with a wavelength between about 250 nm and about 300 nm. In some embodiments, the wavelength is about 254 nm.

In some embodiments, the de-crosslinking comprises irradiating the sample at the wavelength for between about 1 minute and about 10 minutes. In some embodiments, the de-crosslinking comprises irradiating the sample at the wavelength for between about 1 minute and about 3 minutes, or between about 1 minute and about 5 minutes.

In some embodiments, the method comprises detecting the RCP. In some embodiments, detecting the RCP comprises contacting the RCP with one or more detectable probes that hybridize to the RCP. In some embodiments, the one or more detectable probes hybridize to multiple copies of a barcode sequence in the RCP, wherein the barcode sequence in the one or more RCA products is complementary to a barcode sequence in the circular nucleic acid template. In some embodiments, detecting the RCP comprises contacting the RCP with an intermediate probe that hybridizes to the RCP, wherein the intermediate probe comprises a sequence that hybridizes to the RCP and a sequence that hybridizes to one or more detectable probes.

In some embodiments, the detecting step comprises contacting the RCP with a plurality of detectable probes and/or a plurality of intermediate probes in sequential cycles. In some embodiments, the method comprises sequencing all or a portion of the RCP. In some embodiments, the sequencing comprises sequencing-by-synthesis, sequencing-by-binding, or sequencing-by-avidity. In some embodiments, the RCP is detected at a location in the biological sample or a matrix embedding the biological sample.

In some embodiments, the target nucleic acid is a cellular nucleic acid analyte or a product thereof. In some embodiments, the target nucleic acid is associated with a non-nucleic acid analyte. In some embodiments, the target nucleic acid is an oligonucleotide reporter in a labeling agent that binds to the analyte.

In some embodiments, the target nucleic acid is an mRNA. In some embodiments, the target nucleic acid is an RNA fragment. In some embodiments, the target nucleic acid is a probe or probe set associated with a nucleic acid analyte or product thereof in the biological sample.

In some embodiments, the biological sample is a cell or tissue sample comprising cells or cellular components. In some embodiments, the biological sample is non-homogenized. In some embodiments, the biological sample is selected from the group consisting of a formalin-fixed, paraffin-embedded (FFPE) sample, a frozen tissue sample, and a fresh tissue sample.

In some embodiments, the biological sample is permeabilized. In some embodiments, the biological sample is embedded in a matrix. In some embodiments, the matrix comprises a hydrogel. In some embodiments, the biological sample is cleared. In some embodiments, wherein the biological sample is a tissue slice between about 1 μm and about 50 μm in thickness. In some embodiments, the tissue slice is between about 5 μm and about 35 μm in thickness.

In some embodiments, the biological sample is on a substrate.

In some embodiments, provided herein is a kit comprising: a circular probe or a circularizable probe or circularizable probe set, wherein a first nucleic acid strand comprises the circular probe or the circularizable probe, wherein a set of first nucleic acid strands comprises the circularizable probe set, wherein the circularizable probe or circularizable probe set is capable of being circularized upon hybridization to a target nucleic acid, and a second nucleic acid strand comprising a sequence complementary to a sequence of the circular probe, the circularizable probe, or the circularizable probe set, wherein the sequence complementary to a sequence of the circular probe, the circularizable probe, or the circularizable probe set comprises a crosslinkable moiety. In some embodiments, the kit comprises a splint oligonucleotide. In some embodiments, the crosslinkable moiety is a vinylcarbazone-based moiety.

In some embodiments, the crosslinkable moiety is a 3-cyanovinylcarbazole phosphoramidite or a pyranocarbazole phosphoramidite. In some embodiments, the crosslinkable moiety is a 3-cyanovinylcarbazole (^{CNV}K) nucleoside, a 3-cyanovinylcarbazole modified D-threoninol (^{CNV}D), a pyranocarbazole nucleoside (^{PC}X) or a pyranocarbazole modified D-threoninol (^{PCX}D).

In some embodiments, the crosslinkable moiety is a coumarin.

In some embodiments, the second nucleic acid strand is crosslinked to the circular probe or to the circularizable probe or the circularizable probe set.

### BRIEF DESCRIPTION OF THE DRAWINGS

The drawings illustrate certain features and advantages of this disclosure. These embodiments are not intended to limit the scope of the appended claims in any manner.

FIGS. 1A-1B show generation of an example circular nucleic acid template crosslinked to a second nucleic acid strand and contacting with a target nucleic acid within a biological sample. FIG. 1A shows in vitro contacting and subsequent crosslinking of the circular nucleic acid template comprising target-complementary arms and a sequence complementary to the second nucleic acid strand and the second nucleic acid strand comprising one or more crosslinkable bases. FIG. 1B shows hybridization between an example circular nucleic acid template and a target nucleic acid within a biological sample.

FIGS. 2A-2C shows detection of a target nucleic acid within a biological sample using an example circular nucleic acid template. FIG. 2A shows hybridization between an example circular nucleic acid template crosslinked to a second nucleic acid strand and a target nucleic acid within a biological sample followed by ligation of the circular nucleic acid template. FIG. 2B shows contacting of an example polymerase to the second nucleic acid crosslinked to the ligated template followed by generation of an extended nucleic acid priming sequence that stalls at the crosslink. FIG. 2C shows reversal of the crosslink, optional addition of a second polymerase, and subsequent rolling circle amplification and detection of the example ligated template.

FIGS. 3A-3B show example circular nucleic acid templates crosslinked to a second nucleic acid strand comprising an optional 3′ block. FIG. 3A shows hybridization between an example circular nucleic acid template crosslinked to a second nucleic acid strand comprising an optional 3′ block and a target nucleic acid within a biological sample, wherein the target nucleic acid comprises a priming sequence. FIG. 3B shows hybridization between an example circular nucleic acid template crosslinked to a second nucleic acid strand comprising an optional 3′ block and a target nucleic acid within a biological sample, wherein the template is contacted with a primer to enable amplification.

FIG. 4 shows an example circular nucleic acid template comprising one or more crosslinkable bases within target-complementary arms hybridized to a target nucleic acid within a biological sample.

FIG. 5 is an example workflow of analysis of a biological sample (e.g., a cell or tissue sample) using an opto-fluidic instrument, according to various embodiments.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

The ability to detect nucleic acids of interest in situ may reveal spatial expression patterns within a biological sample (e.g., a tissue or a cell) and improve analysis of biological mechanisms involved in both normal and disease states. Rolling circle amplification is one method that provides multiple copies of a sequence of interest (e.g., a barcode sequence or sequence of an endogenous analyte). Using sequencing-based approaches and/or binding of detectably labeled probes to the amplified copies of the sequence of interest provides an amplified signal, which can be detected at a location in a biological sample.

One potential limitation to this approach may be a lack of uniformity between individual signals generated from the detectable probe or plurality of detectable probes within the sample. The lack of uniformity may comprise differences in the non-limiting elements of size, shape, intensity, distribution, and/or differentiation from background noise. For example, if individual signals possess different fluorescent intensities (e.g., one signal is brighter than another signal), then this may result in false negative detection of an individual signal of lower intensity. In certain cases, the lack of uniformity occurs because a polymerase initiates amplification of different circular nucleic acid templates at different times, and/or amplification occurs at different rates. A lack of uniformity across individual signals may therefore negatively affect the ability to map target nucleic acids spatially within the biological sample.

In some cases, different RCA product sizes result from inefficient and/or non-uniform contacting by primers. In some embodiments, a primer is used to facilitate amplification and formation of a product to be detected. In some examples, a circularizable probe that is ligated (e.g., a circular nucleic acid template) after hybridization to a target nucleic acid is contacted by a primer to initiate rolling circle amplification. If primers are contacted with some but not all circular nucleic acid templates upon initiation of amplification, then differential product sizes may result, or some probes may generate no products entirely. Another potential issue may be dissociation of primers already hybridized to circular nucleic acid template. The risk of dissociation may be especially high during washing steps that may take place during detection protocols. In some cases, primers that dissociate from the template prior to amplification may generate smaller products or no products at all, contributing to a lack of signal uniformity or perhaps even false-negative detection.

Another potential cause of product size differences may be unsynchronized amplification. Generation of products may include first contacting the biological sample with polymerase to initiate amplification. If amplification is initiated with only some polymerases bound to a circular nucleic acid template, some probes may generate no products entirely. Another issue may be when polymerases contact the circular nucleic acid template after amplification is initiated. In this instance, products of distinct probes may be different sizes (e.g., one product may be smaller in size than a second product). Without being limited by theory, synchronization of polymerase activity on circular nucleic acid templates may therefore improve uniformity of products to be detected in situ.

Provided herein are methods directed to the use of a crosslinked probe or probe set for ensuring the generation of uniform signals associated with target nucleic acids in situ. In some embodiments, the crosslinked probe or probe set comprises (i) a polynucleotide or set of polynucleotides (e.g., a circularizable probe or probe set) comprising one or more hybridization regions to the target nucleic acid and (ii) a primer, second nucleic acid strand, or nucleic acid priming sequence that hybridizes to a region on the polynucleotide or set of polynucleotides, wherein the primer is crosslinked to the region at one or more bases. In some embodiments, the hybridization and crosslinking of the primer, second nucleic acid strand, or nucleic acid priming sequence to the polynucleotide to form the crosslinked probe or probe set can occur prior to contact with the biological sample (e.g., in vitro). Hybridization between the crosslinked probe or probe set and the target nucleic acid may bring the ends of the probe or probe set into proximity for efficient circularization (e.g., by ligation such as template-mediated ligation), thereby forming a circular nucleic acid template. In some embodiments, forming the circular nucleic acid template comprises ligation. In some embodiments, the ligation is template-mediated ligation. In some embodiments, the circular nucleic acid template crosslinked to the primer, second nucleic acid strand, or nucleic acid priming sequence is then contacted with a polymerase in situ. Without being bound by theory, the presence of the crosslinked primer or nucleic acid priming sequence on the circular nucleic acid template may improve the uniformity of polymerase loading onto the circular nucleic acid template. Initiation of amplification may then take place, resulting in more uniform products of the circular nucleic acid template for subsequent detection and mapping of target nucleic acids in situ.

In some embodiments, after amplification is initiated, polymerases contacted with the primer crosslinked to the circular nucleic acid begin amplifying a product using the circular nucleic acid as a template. In some embodiments, the product is a rolling circle amplification product (RCP). For example, the polymerase uses a circular nucleic acid template as a template for a first extension, thereby generating an extended nucleic acid priming sequence. However, the polymerases may not be able to progress past the one or more crosslinks between the primer and the circular nucleic acid template upon reaching this point during amplification. In some embodiments, the polymerase stalls at the crosslinks while contacted with the circular nucleic acid template. In some embodiments, the polymerase falls off the circular nucleic acid template upon reaching the crosslink. In some embodiments, the biological sample is contacted again with a second polymerase before amplification is continued after the polymerase falls off the circular template. In some embodiments, amplification of a detectable product is only able to continue after the crosslink between the primer and the circular nucleic acid template is removed. In some embodiments, the crosslink is reversed through an intervention. Without being bound by theory, stalling at the crosslink and optional additional contacting of polymerases to the biological sample, in some cases, serve as a point wherein the amplification of a detectable product (e.g., an RCP) can be synchronized. For example, the addition of a reverse crosslinking step to the amplification of a detectable product (e.g., an RCP) ensures that all circular nucleic acid templates are amplified in unison, thereby decreasing differences in the sizes of amplification products and helping ensure uniformity of all related detectable signals.

In another embodiment, a crosslinkable probe or probe set comprises a polynucleotide or set of polynucleotides comprising one or more hybridization regions to the target nucleic acid, wherein the one or more hybridization regions comprise one or more crosslinkable bases. In some embodiments, the polynucleotide or set of polynucleotides is a circularizable probe or circularizable probe set. In some embodiments, a crosslinkable probe or probe set is a single circular molecule (e.g., a circular nucleic acid template) comprising one hybridization region to the target nucleic acid, wherein the one hybridization region comprises one or more crosslinkable bases. In some embodiments, the crosslinkable probe or probe set is contacted with the biological sample and hybridize to the target nucleic acid within the sample. In some embodiments, hybridization between the crosslinkable probe or probe set and the target nucleic acid brings the ends of the probe or probe set into proximity for efficient ligation (e.g., template-mediated ligation), thereby forming the circular nucleic acid template. In some embodiments, the crosslinkable bases of the crosslinkable probe or probe set is crosslinked to the target nucleic acid (e.g., the biological sample is treated with UV to induce crosslinking) following hybridization and prior to ligation. In some embodiments, the crosslinkable bases of the crosslinkable probe or probe set is crosslinked to the target nucleic acid following formation of the circular nucleic acid template (e.g., the biological sample is treated to induce crosslinking after formation of a circular nucleic acid template). In both instances, the crosslinking of the probe to the target nucleic acid takes place in situ to form crosslinked bases that may aid in synchronizing product amplification.

After crosslinking the crosslinkable probe to the target nucleic acid and optionally forming the circular nucleic acid (e.g., by performing a ligation) in situ, the circular nucleic acid may serve as a template for amplification of a detectable product (e.g., a circular nucleic acid template may serve as a template for rolling circle amplification). In some embodiments, forming the circular nucleic acid comprises ligation of a circularizable probe or probe set. In some embodiments, the circular nucleic acid template serves as a template for rolling circle amplification (RCA). In some embodiments, a primer or nucleic acid priming sequence is formed from the target nucleic acid crosslinked to the circular nucleic acid template by cleavage of the target nucleic acid with an endonuclease. For example, if the circular nucleic acid template is DNA and the target nucleic acid is RNA, hybridization forms a DNA: RNA hybrid, and the biological sample can be contacted with an endonuclease which may cleave RNA within the DNA: RNA hybrid. In some embodiments, the endonuclease is RNaseH or a DNAzyme. This cleavage dissociates the hybridized target nucleic acid from the DNA: RNA hybrid except for the one or more regions crosslinked to the circular nucleic acid template. In some embodiments, the one or more regions crosslinked to the circular nucleic acid template serves as a primer for contacting and amplification by polymerases. In some embodiments, the biological sample is contacted by a separate primer that hybridizes to the circular nucleic acid template and the template is amplified by a polymerase.

In some instances, after amplification is initiated, polymerases contacted with the primer, second nucleic acid strand, or nucleic acid priming sequence crosslinked to the circular nucleic acid template begins amplifying a product (e.g., an RCP). However, the polymerases may not be able to progress past the one or more crosslinks between the primer and the circular nucleic acid template upon reaching this point during amplification. This may result in the polymerase stalling at the crosslinks while on the circular nucleic acid template. In some instances, this may result in the polymerase falling off the template, potentially requiring the biological sample be contacted again with polymerases before amplification is continued. In some cases, amplification of a detectable product may only be able to continue after the crosslink between the primer and the circular nucleic acid template is removed (e.g., the biological sample is treated with a UV wavelength to reverse the crosslink). Without being bound by theory, stalling at the crosslink and optional additional contacting of polymerases to the biological sample may therefore serve as another point wherein the amplification of a detectable product (e.g., an RCP) can be synchronized. For example, the addition of a reverse crosslinking step to the amplification of a detectable product (e.g., an RCP) ensures that all circular nucleic acid template are amplified in unison, thereby decreasing differences in the sizes of amplification products and helping ensure uniformity of all detectable signals (e.g., reducing differences in signal intensity).

In some embodiments, the compositions, methods, and systems disclosed herein provide one or more advantages compared to conventional approaches without the use of crosslinked or crosslinkable probes or probe sets. In one aspect, the compositions, methods, and systems disclosed herein are used to control signal amplification, e.g. such that the variation in signal brightness from molecule to molecule is kept relatively small. In one aspect, the compositions, methods, and systems disclosed herein are used to control the physical size or extent of the signal from individual molecules in order to prevent substantial overlap of the signal from physically adjacent molecules. In one aspect, the compositions, methods, and systems disclosed herein improve uniformity of signal formation and signal parameters (e.g., size, shape, intensity, distribution, and/or differentiation from background noise). In one aspect, the compositions, methods, and systems disclosed herein are used to amplify multiple signals in a sample or across multiple different samples of the same type or different types. In some embodiments, the multiple signals are orthogonal and are decoded for multiplex analyte detection in situ.

### II. Methods for Rolling Circle Amplification Comprising Crosslinking and De-Crosslinking

In some aspects, the methods disclosed herein involve nucleic acid hybridization. In some embodiments, hybridization of a circular nucleic acid template herein to a target nucleic acid is detected. In some instances, hybridization of a circularizable probe or probe set to a target nucleic acid is analyzed, wherein the circularizable probe or probe set is circularized after hybridization to the target nucleic acid. In some embodiments, hybridization is achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

In some aspects, provided herein are nucleic acid probes, probe sets, second nucleic acid strands and/or nucleic acid priming sequences. In some embodiments, the nucleic acid priming sequences are exogenous primers. The nucleic acids disclosed herein may comprise any of a variety of entities that can hybridize to another nucleic acid, typically by Watson-Crick base pairing, such as DNA, RNA, LNA, PNA, etc., depending on the application. In some embodiments, a circular probe, or a circularizable probe or probe set comprises a targeting sequence that binds to at least a portion of a target nucleic acid. In some embodiments, the circularizable probe or probe set is able to bind to a specific target nucleic acid (e.g., an mRNA or other nucleic acids as discussed herein). In some embodiments, the circular probe, circular nucleic acid template, or circularizable probe or probe set comprises one or more hybridization regions that bind to a least a portion of a target nucleic acid. In some embodiments, the circularizable probe or probe set bound to the target nucleic acid is ligated to form a circular nucleic acid template. In some embodiments, the circular nucleic acid template is a circular probe.

In some embodiments, a circular nucleic acid template or an amplification product thereof (e.g., an RCA product thereof) is detected or analyzed using sequencing, binding of a probe comprising a detectable label, and/or using intermediate probes or intermediate probe sets that bind to the circular nucleic acid template or amplification product thereof.

### A. Circular Nucleic Acid Template

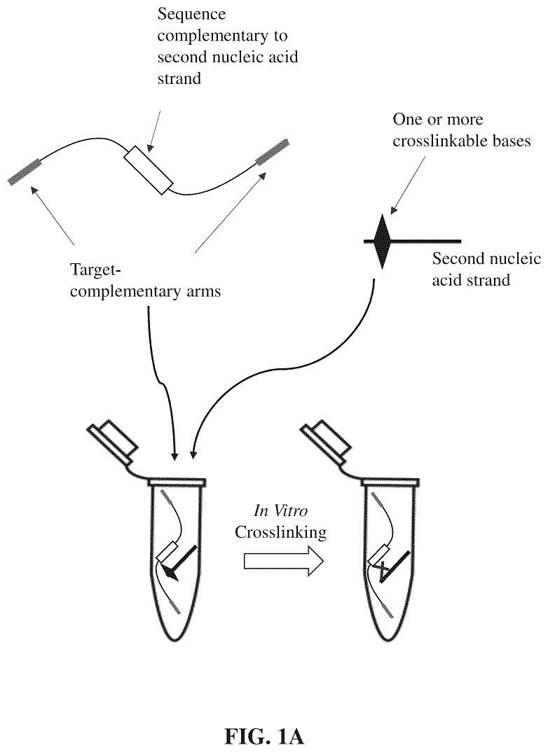

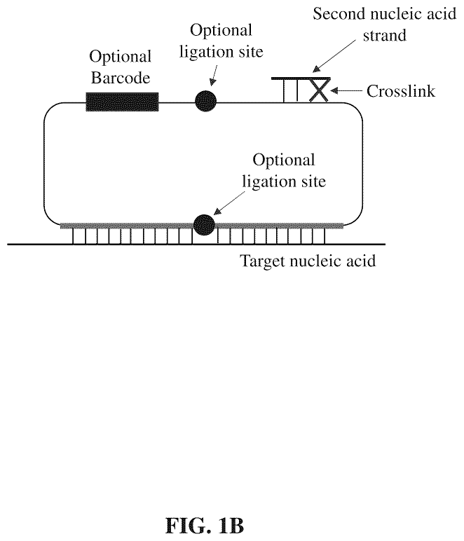

Provided herein are methods comprising extending a nucleic acid priming sequence hybridized to a circular nucleic acid template using a polymerase to generate an extended priming sequence, wherein a first nucleic acid strand comprises the circular nucleic acid template, and the circular nucleic acid template is crosslinked to a second nucleic acid strand. In some cases, the circular template for rolling circle amplification described herein is generated from a circularizable probe or probe set by one or more ligations. In some instances, a circularizable probe is hybridized to a target nucleic acid and circularized by ligation of the 5′ end and 3′ end of the circularizable probe to form the circular nucleic acid template. In some embodiments, the circular nucleic acid template is generated by ligating a padlock probe prior to (a). In some embodiments, the circular nucleic acid template is generated by ligating a circularizable probe set at two or more ligation sites to form a circular nucleic acid template. In some embodiments, the circularizable probe set comprises a first nucleic acid molecule and a second nucleic acid molecule. In some instances, a 5′ end of the first nucleic acid molecule is ligated to the 3′ end of the second nucleic acid molecule using the target nucleic acid as a template. In some embodiments, the 3′ end of the first nucleic acid is ligated to the 5′ end of the second nucleic acid molecule in a second ligation. In some embodiments, the second ligation is performed using a splint oligonucleotide that hybridizes to the 5′ end of the first nucleic acid molecule and to the 3′ end of the second nucleic acid molecule as a template. FIG. 1B illustrates an example of how one or more ligation sites (e.g., one, two, or more ligation sites) are used to generate a circular nucleic acid template from a circularizable probe or probe set.

In some embodiments the first nucleic acid strand comprises a circularizable probe from which the circular nucleic acid template is generated. In some embodiments, a set of first nucleic acid strands comprises a circularizable probe set from which the circular nucleic acid template is generated. In some embodiments, the circularizable probe or the circularizable probe set is circularized by ligation. In some embodiments, the ligation is template-mediated ligation, wherein a target nucleic acid molecule hybridized to the circularizable probe or the circularizable probe set serves as the template for the ligation. In some embodiments, the ligation is template-mediated ligation, wherein a splint oligonucleotide hybridized to the circularizable probe or the circularizable probe set serves as the template for ligation. In some embodiments, the circularizable probe set is circularized using two or more ligations.

In some embodiments, the first nucleic acid strand comprises a target nucleic acid sequence or a product thereof. In some embodiments, the target nucleic acid is RNA. In some embodiments, the target nucleic acid is reverse-transcribed to form a cDNA molecule. In some embodiments, the target nucleic acid is reverse-transcribed by contacting the biological sample with an oligonucleotide that hybridizes to the target nucleic acid, a reverse transcriptase, and free nucleotides. In some embodiments, the oligonucleotide hybridizes to the target nucleic acid and is extended by the reverse transcriptase using the free nucleotides to form the cDNA molecule. In some embodiments, the cDNA molecule derived from the target nucleic acid is circularized to form the circular nucleic acid template. In some embodiments, circularization of the cDNA molecule comprises ligation of the ends of the cDNA molecule to each other using a ligase.

In some embodiments, the circularizable probe or probe set is crosslinked to the nucleic acid priming sequence before circularizing the probe or probe set to form the circular nucleic acid template. In some embodiments, the circularizable probe or probe set is crosslinked to the nucleic acid priming sequence after circularizing the nucleic acid probe or probe set to generate the circular nucleic acid template.

In some embodiments, the circular nucleic acid template or circularizable probe or probe set for generating the circular nucleic acid template comprises one, two, three, four, five, or more crosslinkable moieties. In some embodiments, the circular nucleic acid template or circularizable probe or probe set for generating the circular nucleic acid template comprises two or more crosslinkable moieties. Examples of crosslinkable moieties include but are not limited to those described in Section II-C. In some embodiments, the circular nucleic acid template or circularizable probe or probe set for generating the circular nucleic acid template comprises one or more crosslinkable moieties comprised by bases within its sequence. In some embodiments, the circular nucleic acid template or circularizable probe or probe set for generating the circular nucleic acid template comprises at least 1, 2, 3, 4, 5, 6, or more crosslinkable moieties. In some embodiments, the circular nucleic acid template or circularizable probe or probe set for generating the circular nucleic acid template comprises anywhere between 1-10 crosslinkable moieties. In some embodiments, the circular nucleic acid template or circularizable probe or probe set for generating the circular nucleic acid template comprises 1-2 crosslinkable moieties. In some embodiments, at least one of the one or more crosslinkable moieties is located at the 5′ terminal base of the circularizable probe or probe set for generating the circular nucleic acid template. In some embodiments, at least one of the one or more crosslinkable moieties is located at the 3′ terminal base of the circularizable probe or probe set for generating the circular nucleic acid template. In some embodiments, at least one of the one or more crosslinkable moieties is located in the 5′ hybridization region in the circularizable probe or probe set for binding to the target nucleic acid. In some embodiments, at least one of the one or more crosslinkable moieties is located at the 3′ hybridization region in the circularizable probe or probe set for binding to the target nucleic acid.

In some embodiments, the circular nucleic acid template does not comprise a crosslinkable moiety. For example, in some cases, the second nucleic acid strand comprises one or more crosslinkable moieties that can be crosslinked to the circular nucleic acid template or circularizable probe or probe set upon hybridization and irradiation with one or more wavelengths of light.

In some embodiments, the circularizable probe or probe set is capable of DNA-templated ligation, for example ligation templated by a cDNA molecule. See, e.g., U.S. Pat. No. 8,551,710, the content of which is herein incorporated by reference in its entirety. In some embodiments, provided herein is a probe or probe set capable of RNA-templated ligation. See, e.g., U.S. Pat. Pub. 2020/0224244, the content of which is herein incorporated by reference in its entirety.

In some embodiments, the target nucleic acid for binding of a circular nucleic acid template, a circularizable probe, or a circularizable probe set is an analyte or a product thereof. In some embodiments, the analyte is DNA. In some embodiments, the analyte is genomic DNA. In some embodiments, the analyte is cDNA. In some embodiments, the analyte is RNA. In some embodiments, the analyte is mRNA. In some embodiments, the analyte is a reporter oligonucleotide comprised on a labeling agent. In some embodiments, the product thereof is an amplification product of the target nucleic acid molecule. In some embodiments, the target nucleic acid molecule or a product thereof is directly contacted by the circular nucleic acid template, the circularizable probe, and/or the circularizable probe set. In some embodiments, the target nucleic acid or a product thereof comprises a region for direct hybridization to the circular nucleic acid template, the circularizable probe, or the circularizable probe set. In some embodiments, the target nucleic acid or a product thereof is indirectly contacted by the circular nucleic acid template, the circularizable probe, or the circularizable probe set through an intermediate probe or intermediate probe set. In some embodiments, the intermediate probe or intermediate probe set comprises one or more regions that hybridize to the target nucleic acid molecule and one or more regions that hybridize to the circular nucleic acid template, the circularizable probe, or the circularizable probe set.

In some embodiments, the target nucleic acid molecule is a probe that directly or indirectly binds to an analyte or product thereof. In some embodiments, the target nucleic acid molecule is a primary probe that hybridizes to a nucleic acid analyte in the biological sample.

In some embodiments, the circular nucleic acid template comprises a barcode, or a set of barcodes that correspond to a target nucleic acid. In some cases, the target nucleic acid is a nucleic acid analyte. In some embodiments, the target nucleic acid is associated with a nucleic acid analyte by binding directly or indirectly to the analyte (e.g., via hybridization of one or more probes to a nucleic acid analyte, or via binding of a labeling agent to a non-nucleic acid analyte such as a protein). In some cases, binding of the circular nucleic acid template directly or indirectly to the target nucleic acid associates the barcode or set of barcodes of the circular nucleic acid template with the target nucleic acid, and optionally with an analyte bound directly or indirectly to the target nucleic acid. In some embodiments, the circular nucleic acid template is used in an amplification reaction (e.g., RCA). In some embodiments, an RCA product generated from the circular nucleic acid template comprises multiple copies of a sequence complementary to the barcode or set of barcodes. In some cases, detecting the RCA product comprises detecting the complement(s) of the barcode or barcodes in the RCA product. In some embodiments, the barcode or barcodes are associated with the analyte, and detecting the complement(s) of the barcode or barcodes in the RCA product allows detection of the analyte.

In some embodiments, the circular nucleic acid template comprises one or more barcodes. In some embodiments, the barcodes correspond to a target nucleic acid to which the circular nucleic acid template is configured to bind directly or indirectly. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In some embodiments, the methods provided herein include analyzing the barcodes (or complements thereof) by sequential hybridization and detection with a plurality of detectably labelled probes.

In some embodiments, barcode sequences or complements thereof are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4N complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences. Detection of barcode sequences can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, each of which are incorporated herein by reference in its entirety.

In some embodiments, the circularizable probe is a single nucleic acid strand that is circularized upon hybridization to one or more other nucleic acid molecules. In some embodiments, the circularizable probe comprises in the 5′ to 3′ direction or the 3′ to 5′ direction: a first hybridization region that hybridizes to a corresponding first sequence in a target nucleic acid; optionally a detectable sequence (e.g., a barcode region comprising one or more barcode sequences or complements thereof); and a second hybridization region that hybridizes to a corresponding second sequence in a target nucleic acid. In some embodiments, the first sequence and second sequences are adjacent, and the ends of the circularizable probe are ligated without gap-filling. In some embodiments, the ends of the circularizable probe are ligated with gap-filling. In some embodiments, the target nucleic acid comprises the nucleic acid priming sequence.

In some embodiments, the probe is circularized to form a circular nucleic acid template before being crosslinked to the target nucleic. In some embodiments, the probe is circularized to form a circular nucleic acid template after being crosslinked to the target nucleic acid In some embodiments, the method comprises contacting the biological sample with a separate primer that hybridizes to the circular nucleic acid template formed from a circularizable probe, and the circular nucleic acid template further comprises a primer-binding sequence that hybridizes to the separate primer. In some embodiments, the primer-binding sequence is crosslinked to the primer. In some embodiments, the circularizable probe or probe set is a circularizable probe set comprising two or more nucleic acid molecules (e.g., probes) that are ligated together to generate the circular nucleic acid template. In some embodiments, the circularizable probe set comprises a first nucleic acid probe and a second nucleic acid probe. In certain embodiments, the first nucleic acid probe comprises in the 5′ to 3′ direction: a first hybridization region that hybridizes to a corresponding first sequence in a target of the probe; an optional detectable sequence (e.g., a barcode region comprising one or more barcode sequences or complements thereof); and a first splint complementary sequence; and the second nucleic acid probe comprises in the 5′ to 3′ direction: a second splint complementary sequence; a second nucleic acid strand hybridizing region; and a second hybridization region that hybridizes to a corresponding second sequence in a target of the probe. In some embodiments, the first sequence and second sequences in the target nucleic acid are adjacent, and the ends of the first probe and the second probe are ligated without gap-filling. In some embodiments, the ends of the first probe and the second probe are ligated with gap-filling.

In some embodiments, the circularizable probe set is contacted with a splint oligonucleotide. In some embodiments, the splint oligonucleotide comprises a first sequence complementary with the first splint complementary region comprised by the first nucleic acid probe, and a second sequence complementary with the second splint complementary sequence comprised by the second nucleic acid probe. In some embodiments, the splint oligonucleotide hybridized to the circularizable probe set facilitates ligation of the probe set at the junction between the first and second nucleic acid probes. In some embodiments, splint-mediated ligation of the probe set occurs after the probe set has hybridized to the target of the probe. In some embodiments, splint-mediated ligation of the probe set occurs before the probe set has hybridized to the target of the probe. In some embodiments, the splint is the second nucleic acid strand which is crosslinked to the circular nucleic acid template.

In some embodiments, a the circular nucleic acid template (e.g., generated from a circular probe or circularizable probe or probe set) disclosed herein comprises 1, 2, 3, 4, 5, 6, 7, 8, 9, 10, 11, 12, 13, 14, 15 or more, 20 or more, 32 or more, 40 or more, or 50 or more barcode sequences. The barcode sequences may be positioned anywhere within the circularizable probe or probe set. If more than one barcode sequences are present, the barcode sequences may be positioned next to each other, and/or interspersed with other sequences. In some embodiments, two or more of the barcode sequences also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence is no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology is less than 10%, less than 8%, less than 7%, less than 6%, less than 5%, less than 4%, less than 3%, less than 2%, or less than 1%. In some embodiments, the homology is less than 20, 19, 18, 17, 16, 15, 14, 13, 12, 11, 10, 9, 8, 7, 6, 5, 4, 3, or 2 bases. In some embodiments, the bases are consecutive bases.

In some embodiments, the number of distinct barcode sequences in a population of circularizable probe or probe sets is less than the number of distinct targets (e.g., nucleic acid analytes and/or protein analytes) of the circularizable probe or probe sets, and yet the distinct targets are still uniquely identified from one another. In some embodiments, the circularizable probe or probe set is encoded with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes each comprise the same number of barcode sequences. In some embodiments, there are different numbers of barcode sequences present on the various templates (e.g., in the population of nucleic acid probes). In some embodiments, the barcode sequences or any subset thereof in the population of circularizable probe or probe sets are independently and/or combinatorially detected and/or decoded.

In some embodiments, the circularizable probe or probe set is contacted directly to the target analyte within the biological sample. In some embodiments, the circularizable probe or probe set comprises a first hybridization region that hybridizes to a corresponding first sequence in the target of the template and a second hybridization region that hybridizes to a corresponding second sequence in the target of the template. In some embodiments, the circularizable probe is a linear polynucleotide comprising the first and second hybridization regions on the 5′ and 3′ ends of the linear polynucleotide, respectively. In some embodiments, hybridization of the linear polynucleotide to the target analyte forms the circularizable probe, wherein template-mediated ligation forms the circular nucleic acid template. In some embodiments, the circularizable probe set is a set of two linear polynucleotides comprising the first and second hybridization regions on either the 5′ and/or 3′ ends of each linear polynucleotide. In some embodiments, hybridization of the two linear polynucleotides to the target analyte forms the circularizable probe set. In some embodiments, both template-mediated and splint-mediated ligation form the circular nucleic acid template from the two linear polynucleotides/the circularizable probe set.

### B. Second Nucleic Acid Strand

Disclosed herein are methods comprising extending a nucleic acid priming sequence hybridized to a circular nucleic acid template using a polymerase to generate an extended priming sequence, wherein a first nucleic acid strand comprises the circular nucleic acid template, and the circular nucleic acid template is crosslinked to a second nucleic acid strand; de-crosslinking the circular nucleic acid template from the second nucleic acid strand; and extending the extended priming sequence to generate a rolling circle amplification product (RCP). In some embodiments, the second nucleic acid strand comprises the nucleic acid priming sequence. In some embodiments, the nucleic acid priming sequence is in a separate nucleic acid molecule. In some aspects, the first nucleic acid strand and the second nucleic acid strand are two separate nucleic acid molecules. In some aspects, the first nucleic acid strand and the second nucleic acid strand are contacted with the biological sample separately. In some aspects, the second nucleic acid strand is contacted with the biological sample when the first nucleic acid strand is in the biological sample. In some aspects, the first nucleic acid strand and the second nucleic acid strand are crosslinked prior to being contacted with the biological sample. In some embodiments, the nucleic acid priming sequence is in an endogenous nucleic acid in the biological sample. In some embodiments, the nucleic acid priming sequence is comprised in an exogenous nucleic acid. In some cases, the second nucleic acid strand is a separate nucleic acid molecule from the nucleic acid priming sequence.

In some embodiments, a set of first nucleic acid strands and the second nucleic acid strand are contacted with the biological sample separately. In some aspects, the second nucleic acid strand is contacted with the biological sample when the set of first nucleic acid strands is in the biological sample. In some aspects, the set of first nucleic acid strands and the second nucleic acid strand are crosslinked prior to being contacted with the biological sample.

In some embodiments, the second nucleic acid strand comprises one, two, three, four, five, or more crosslinkable moieties. In some embodiments, the second nucleic acid strand comprises two or more crosslinkable moieties. Examples of crosslinkable moieties include but are not limited to those described in Section II-C.

The second nucleic acid strand may comprise in some aspects any of a variety of entities that can hybridize to a nucleic acid, typically by Watson-Crick base pairing, such as DNA, RNA, LNA, PNA, BNA, etc., depending on the application. The second nucleic acid strand typically contains a sequence complementary to the second nucleic acid strand hybridizing region of the circular nucleic acid template (e.g., circular probe or generated from the circularizable probe or probe set). In some cases, the second nucleic acid strand binds to a specific circular nucleic acid template, circularizable probe or probe set. For example, in some cases, the second nucleic acid strand binds to a specific circular nucleic acid template or circularizable probe or probe set of a plurality of circularizable templates or circularizable probes or probe sets that are contacted with the biological sample. In some embodiments, the second nucleic acid strand hybridizing region is common among a plurality of circular nucleic acid templates or circularizable probes or probe sets in the biological sample. In some embodiments, a first plurality of second nucleic acid strands comprise a first common sequence for binding a first plurality of circular nucleic acid templates in the biological sample and a second plurality of second nucleic acid strands comprise a second common sequence for binding a second plurality of circular nucleic acid templates in the biological sample. In some embodiments, the first and second plurality of second nucleic acid strands comprise different crosslinkable moieties. In some embodiments, the second nucleic acid strand is an RNA molecule. In some embodiments, the RNA molecule is an endogenous RNA molecule to a biological sample. In some embodiments, the RNA molecule is an exogenous RNA molecule to a biological sample. In some embodiments, the second nucleic acid strand is a DNA molecule. In some embodiments, the second nucleic acid strand comprises a hybridizing region for binding to the circular nucleic acid template and an additional hybridization region for binding to the target nucleic acid hybridized to the circular nucleic acid template. In some embodiments, the second nucleic acid strand hybridizing region is a primer binding region.

In some embodiments, the second nucleic acid strand is at least 5-100 nucleotides in length. In some embodiments, the second nucleic acid strand is 5-20 nucleotides in length, 20-40 nucleotides in length, 20-60 nucleotides in length, 20-80 nucleotides in length, or 20-100 nucleotides in length. In some embodiments, the second nucleic acid strand is 10-30 nucleotides in length. In some embodiments, the second nucleic acid strand is 15-35 nucleotides in length. In some embodiments, the second nucleic acid strand is longer than 100 nucleotides in length.

In some embodiments, the second nucleic acid strand comprises one or more crosslinkable moieties comprised by bases within its sequence. In some embodiments, the second nucleic acid strand comprises at least 1, 2, 3, 4, 5, 6, or more crosslinkable moieties. In some embodiments, the second nucleic acid strand comprises anywhere between 1-10 crosslinkable moieties. In some embodiments, the second nucleic acid strand comprises 1-2 crosslinkable moieties. In some embodiments, at least one of the one or more crosslinkable moieties is located at the 5′ terminal base of the second nucleic acid strand.

In some embodiments, the second nucleic acid strand is an exogenous nucleic acid that is contacted with the biological sample. In some embodiments, the second nucleic acid strand comprises an attachment moiety. In some embodiments, the second nucleic acid strand is attached to the biological sample or to a matrix embedding the biological sample. In some embodiments, the second nucleic acid strand is attached to the biological sample or to a matrix embedding the biological sample at the attachment moiety. In some embodiments herein, the attachment moiety is a 5′ acrydite.

In some embodiments, the circularizable probe or probe set comprises one or more crosslinkable moieties. In some embodiments, the circularizable probe or probe set comprises one or more nucleotide residues functionalized with a crosslinkable moiety. In some embodiments, bases within the second nucleic acid strand hybridizing region of the circularizable probe or probe set comprise the one or more crosslinkable moieties. In some embodiments, bases within the first and/or second hybridization region that hybridizes to a corresponding first and/or sequence in a target nucleic acid of a circular nucleic acid probe or a circularized probe or probe set comprise the one or more crosslinkable moieties.

In some embodiments, the second nucleic acid strand hybridized and/or crosslinked to the circularizable probe or probe set is capable of binding a polymerase. In some embodiments, the polymerase binds the second nucleic acid strand at any region along the 5′ to 3′ length of the second nucleic acid strand. In some embodiments, the polymerase binds to the second nucleic acid strand at the 5′ end of the strand. In some embodiments, the polymerase binds to the second nucleic acid strand at the 3′end of the strand. In some embodiments, the polymerase extends the second nucleic acid strand hybridized to the circular nucleic acid template, thereby forming an extended priming sequence. In some embodiments, the polymerase forms the extended priming sequence by incorporating nucleotides into the second nucleic acid strand contacted with the biological sample. In some embodiments, the polymerase forms the extended priming sequence using the circular nucleic acid template for sequence-complementary hybridization (e.g., Watson-Crick base pairing).

In some embodiments, the second nucleic acid strand is incapable of being extended by a polymerase. In some embodiments, the second nucleic acid strand comprises a 3′ block moiety. In some embodiments, the 3′ block moiety is an irreversible terminating group. In some embodiments, the irreversible terminating group is a 3′ dideoxynucleotide. In some embodiments, the 3′ block moiety is a reversible terminating group. In some embodiments, the reversible terminating group is an azidomethyl group, an amino group, a nitrobenzyl group, or an allyl group.

### C. Crosslinkable Moieties, Crosslinking, and De-crosslinking

In some aspects, a circular nucleic acid template (e.g., a circular probe or a template generated from a circularizable probe or probe set) is crosslinked to a second nucleic acid strand according to the methods described herein using a crosslinkable moiety. In some instances, a first nucleic acid strand comprises the circular nucleic acid template. In some instances, a first nucleic acid strand comprises a target nucleic acid bound by (e.g., hybridized to) the circular nucleic acid template. In some embodiments, a linear nucleic acid molecule (e.g., a first nucleic acid strand) is crosslinked to a second nucleic acid strand prior to forming the circular nucleic acid template. In some embodiments, the linear nucleic acid molecule is a circularizable probe or part of a circularizable probe set. In some aspects, a circular nucleic acid template is crosslinked to a second nucleic acid strand using a reversible crosslinkable moiety. In some embodiments, a circularizable probe or probe set is crosslinked to a second nucleic acid strand using a reversible crosslinkable moiety. In some embodiment, provided herein is a complex comprising a first nucleic acid strand (e.g., a circular nucleic acid template) reversibly crosslinked to a second nucleic acid strand using a crosslinkable moiety, wherein the first nucleic acid strand is used as template for an extension reaction.

In some embodiments, the one or more crosslinkable moieties is a vinylcarbazone-based moiety. In some embodiments, the crosslinkable moiety is a 3-cyanovinylcarbazole phosphoramidite or a pyranocarbazole phosphoramidite. In some embodiments, the crosslinkable moiety is a 3-cyanovinylcarbazole (^{CNV}K) nucleoside, a 3-cyanovinylcarbazole modified D-threoninol (^{CNV}D), a pyranocarbazole nucleoside (^{PC}X) or a pyranocarbazole modified D-threoninol (^{PCX}D). In some embodiments the crosslinkable moiety is a coumarin.

In some embodiments, the second nucleic acid strand is hybridized to the circularizable probe or circularizable probe set at the second nucleic acid strand hybridizing region. In some embodiments, the second nucleic acid strand is crosslinked to the circularizable probe or circularizable probe set at bases hybridized to the second nucleic acid strand hybridizing region, wherein the bases comprise the one or more crosslinkable moieties.

In some embodiments, the crosslinking occurs before contacting the circularizable probe or probe set to the biological sample. In some embodiments, the crosslinking occurs by irradiating a mixture comprising the second nucleic acid strand and the circularizable probe or probe set with UV light prior to contacting the biological sample. In some embodiments, the mixture is irradiated using a 350-400 nm wavelength of light. In some embodiments, the mixture is irradiated using a 360-370 nm wavelength of light. In some embodiments, the mixture is irradiated for from about 1 minute to about 10 minutes.

In some embodiments, crosslinking is achieved by contacting a circular probe or circularizable probe or probe set hybridized to the second nucleic acid strand with a crosslinkable intercalating agent such as a psoralen. In some embodiments, the crosslinking is performed in vitro. Psoralens are molecules that can intercalate nucleic acids (e.g., DNA) and upon irradiation can form covalent bonds with pyrimidines (C/T/U). In the absence of irradiation, psoralens bind non-covalently similarly to any other intercalating agent, and covalent crosslinking to nucleic acid depends on irradiation. In some embodiments, the crosslinkable moiety comprises 8-methoxypsoralen (8-MOP), 5-methoxypsoralen (5-MOP), or 4,5′,8-trimethylpsoralen (TMP), or any combination thereof.

In some embodiments, the second nucleic acid strand comprises a crosslinkable moiety. In some embodiments, the crosslinkable moiety is photo-crosslinkable. In some cases, the second nucleic acid strand comprises 1, 2, 3, 4, 5, or more of the crosslinkable moiety (e.g., 2, 3, 4, 5, or more nucleotide residues functionalized with a crosslinkable moiety). In some embodiments, the crosslinkable moiety is an intercalating photoactive moiety within a nucleic acid duplex formed between the circular nucleic acid template and the second nucleic acid strand. In some embodiments, the nucleic acid duplex is formed before one or more ligations to generate the circular nucleic acid template from a circularizable probe or probe set. In some embodiments, the crosslinkable moiety is a chemically modified nucleic acid which is light-responsive. In some embodiments, irradiation with light triggers photochemical reactions that result in chemical ligation, reversal of chemical ligation, or nucleic acid dehybridization. In some embodiments, the crosslinkable moiety comprises a cyclobutane pyrimidine modification. In some embodiments, the crosslinkable moiety comprises a vinyl modification, such as cyanovinyl carbazole or derivative thereof. In some embodiments, the crosslinkable moiety comprises a carbazole modification. In some embodiments, the crosslinkable moiety comprises 5-carboxyvinyl-2′-deoxyuridine. In some embodiments, the crosslinkable moiety comprises a p-carbamoylvinyl phenol nucleoside. In some embodiments, the crosslinkable moiety comprises a cinnamate. In some embodiments, the crosslinkable moiety comprises an azobenzene or derivative thereof. Additional crosslinkable moieties can include but are not limited to those described in De Fazio et al., “Chemically modified nucleic acids and DNA intercalators as tools for nanoparticle assembly,” Chem. Soc. Rev., 2021, 50, 13410, the content of which is herein incorporated by reference its entirety.

In some embodiments, crosslinking is achieved through click reaction chemistry. Any suitable click reaction and click reactive groups may be used. In some embodiments, the click chemistry reaction consists of the condensation of organic azides with alkyne groups to form 1,2,3-triazole linkages. In some embodiments, crosslinking is achieved through copper catalyzed azide alkyne cycloaddition (CuAAC). In some embodiments, crosslinking is achieved through metal-free click chemistry. In some embodiments, crosslinking is achieved through strain promoted azide alkyne cycloaddition (SPAAC). In some embodiments, the SPAAC reaction involves the cycloaddition between a strained cyclooctyne and an organic azide.

In some embodiments, the acceptor moieties comprise dibenzocyclooctyne (DBCO)- or alkyne-modified bases and the detectable moieties comprise an azide moiety. In some embodiments, the acceptor moieties comprise azide-modified bases and the detectable moieties comprise a DBCO or alkyne moiety. In some embodiments, the branch points on the first nucleic acid strand and second nucleic acid strand comprise an azide-modified base. In some embodiments, the acceptor moiety on the second nucleic acid strand and detectably labeled probe comprise an dibenzocyclooctyne (DBCO)- or alkyne-modified base. In some embodiments, covalently attaching the DBCO or alkyne modified bases and azide moieties comprises performing a copper-free or copper-catalyzed click chemistry reaction. In some embodiments, the covalently attaching the detectable moieties to the second nucleic acid strand comprises performing a copper-free or copper-catalyzed click chemistry reaction.

In some embodiments, activation of the crosslinkable moiety is light driven and performed in aqueous solution. In some embodiments, crosslinking strands of nucleic acid molecules comprise at least one photo-reactive nucleobase. In some embodiments, the crosslinkable moiety is a photo-reactive nucleobase. In some embodiments, the photo-reactive nucleobase is any modified nucleobase that is capable of forming a crosslink with another nucleobase in an opposite hybridized strand in the presence of light. In some embodiments, the photo-reactive nucleobase is a modified pyrimidine or purine nucleobase. In some embodiments, the photo reactive nucleobase comprises a vinyl, acrylate, N-hydroxysuccinimide, amine, carboxylate, or thiol chemical group. In some embodiments, the photo-reactive nucleobase comprises a bromo-deoxyuridine. Exemplary photoreactive crosslinkable moieties and photoreactive nucleotides are described, for example, in Elskens and Madder RSC Chem. Biol., 2021, 2, 410 422, the content of which is herein incorporated by reference in its entirety.

In some embodiments, the crosslinkable moiety comprises a reactive chemical group that requires light activation to initiate crosslinking. In some embodiments, the chemical group comprises, for example, an aryl azide, azido-methyl-coumarin, benzophenone, anthraquinone, certain diazo compounds, diazirine, or a psoralen derivative. In some embodiments, the psoralen derivative is selected from the group consisting of 8-methoxypsoralen (8-MOP), 5-methoxypsoralen (5-MOP), and 4,5′,8-trimethylpsoralen (TMP).

In some embodiments, the crosslinkable moiety comprises a cyanovinylcarbazole moiety. In some embodiments, the crosslinkable moiety is a vinylcarbazone-based moiety. In some embodiments, the crosslinkable moiety comprises a 3-cyanovinylcarbazole (^{CNV}K) nucleoside or 3-cyanovinylcarbazole modified D-threoninol (^{CNV}D). In some embodiments, the crosslinkable moiety comprises 3-cyanovinylcarbazole phosphoramidite. In some embodiments, the crosslinkable moiety comprises a pyranocarbazole. In some embodiments, the crosslinkable moiety comprises a pyranocarbazole (^{PC}X) modified nucleoside or a pyranocarbazole with a D-threoninol instead of a 2′-deoxyribose backbone (^{PCX}D). In some embodiments, the crosslinkable moiety comprises 5-carboxyvinyl-2′-deoxyuridine. In some embodiments, the crosslinkable moiety comprises a psoralen or a coumarin. In some embodiments, the photoreactive nucleotides have been attached to the oligonucleotide via a linker. In some embodiments, the linker is a disulfide linker. In some embodiments, the crosslinkable moiety is a photoreactive nucleotide comprising a universal base. In some embodiments, the crosslinkable moiety is 5-bromo-2′-deoxyuridine (BrdU) or 5-bromo-2′-deoxycytidine (BrdC).

In some embodiments, the crosslinkable moiety is a pyranocarbazole (^{PC}X) modified nucleoside. The ^{PC}X crosslinking base displays high crosslinking efficiency with a thymine (T) base or a cytosine (C) base that is positioned adjacent to the base on the complementary strand and is directly incorporated into the DNA hybridization domain itself as a base substitution. In some embodiments, a crosslinking reaction is performed using 400 nm wavelength of light and is completed within about 10 seconds. In some embodiments, a crosslinking reaction is completed within 0.1, 0.25, 0.5, 1, 5, or 10 seconds. In some embodiments, a crosslinking reaction is completed within 0.5, 1, 5, 10, 20, 30, 40, 50, or 60 minutes. In some embodiments, a crosslinking reaction is completed within 0.5, 1, 2, 3, 4, or 5 minutes. In some embodiments, a crosslinking reaction has negligible effects on bases that neighbor the photoreactive nucleobase. In some embodiments, other photochemical nucleic acid crosslinking agents, including psoralen is used in combination with the photoreactive nucleobases disclosed herein. In some embodiments, a photo-induced crosslink is reversible. In some embodiments, a ^{PC}X crosslink is reversed when exposed to 312 or 305 nm UV light.

In some embodiments, other photochemical nucleic acid crosslinking agents, including psoralen and psoralen derivatives are used as crosslinkable moieties. Psoralen and psoralen derivatives can be light-activated with a UV-A of 365 nm. In some embodiments, the psoralen derivative is a psoralen modified nucleoside. Psoralens react with nearby pyrimidine residues. A variety of nucleosides modified with psoralen or psoralen derivatives may be used. For example, click chemistry using a psoralen azide and a nucleosidic alkyne derivative can be used to generate a variety of photoreactive nucleotides. The psoralen can be connected to the nucleotide via a linker, such as a phosphoramidite. Exemplary psoralen derivatives comprising phosphoramidite include but are not limited to 6-[4′-(Hydroxymethyl)-4,5′,8-trimethylpsoralen]-hexyl-1-O-(2-cyanoethyl)-(N,N-diisopropyl)-phosphoramidite and 2-[4′-(hydroxymethyl)-4,5′,8-trimethylpsoralen]-ethyl-1-O-(2-cyanoethyl)-(N,N-diisopropyl)-phosphoramidite. In some embodiments, the psoralen or psoralen derivative is conjugated to position 5 of a uridine or pseudouridine (optionally via a linker). In some cases, the psoralen or psoralen derivative is conjugated to the 2′ position of a sugar ring of a uridine or pseudouridine (optionally via a linker). In some embodiments, the psoralen derivative is an amine-reactive derivative, which is conjugated to an amine-modified nucleotide (e.g., an aminoallyl uridine or pseudouridine nucleotide).

In some embodiments, a psoralen-crosslink (e.g., an interstrand crosslink between the circular nucleic acid template and the second nucleic acid strand) is reversed (de-crosslinked) when exposed to 254 nm light. In some embodiments, the crosslinkable moiety comprises a C2′ psoralen modification. The crosslinkable moiety can comprise a 5′ psoralen derivative, and can be at 5′ end of the oligonucleotide. The structure of two exemplary psoralen-modified oligonucleotides (one 5′ modified nucleoside on the left, and one C2′ modified nucleoside on the right) are shown below:

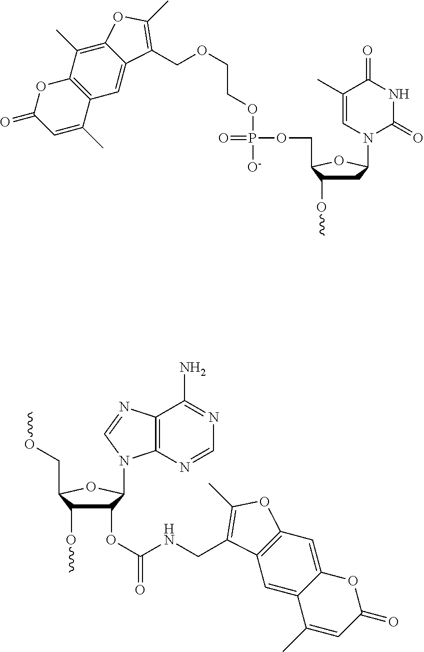

In some embodiments, the crosslinkable moiety is or is linked to a photoactivatable nucleotide, wherein the photoactivatable nucleotide is a universal base such as a pseudouridine modified with a photoreactive moiety (e.g. a psoralen).

In some embodiments, when the oligonucleotide comprises ^{CNV}K, rapid photo cross-linking to pyrimidines in the complementary strand (DNA or RNA) is induced at one wavelength and rapid reversal of the cross-link is possible at a second wavelength if desired. Neither wavelength has the potential to cause significant DNA damage and neither interfere with the wavelengths used to excite the fluorophores used during subsequent analysis, such as decoding barcode sequences in situ. Once cross-linked, the UV melting temperature of the duplex may be raised by around 30° C./^{CNV}K moiety relative to the duplex before irradiation and inter-strand crosslinking. The structure of an exemplary 3-cyanovinylcarbazole phosphoramidite is shown below:

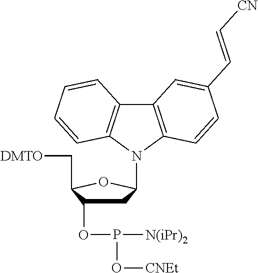

### 5′-O-(4,4′-Dimethoxytrityl)-1′-(3-cyanovinylcarbazol-9-yl)-2′-deoxy-β-D-ribofuranosyl-3′-[(2-cyanoethyl)-(N,N-diisopropyl)]-phosphoramidite

The ^{CNV}K crosslinking base displays high crosslinking efficiency with a thymine (T) base that is positioned adjacent to the base on the opposite hybridized strand in the target nucleic acid (e.g., the complementary strand) (Ultrafast reversible photo-cross-linking reaction: toward in situ DNA manipulation. Org. Lett. 10, 3227-3230 (2008)) and can be directly incorporated into the DNA hybridization domain itself as a base substitution, as shown below in light-directed reaction between a ^{CNV}K base modification and a thymine base to produce a crosslinked nucleic acid.

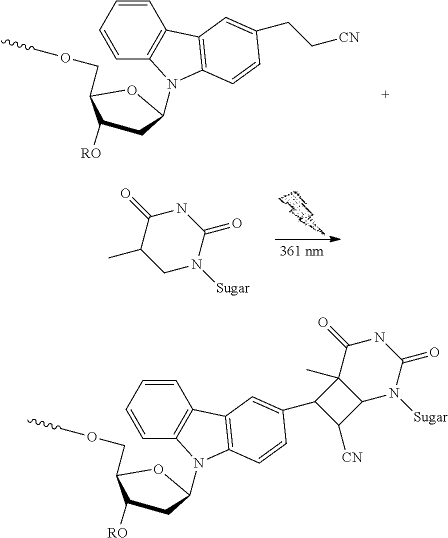

In some embodiments, a crosslinking reaction is performed using 365 nm wavelength of light and is completed within about 1 second. In some embodiments, a crosslinking reaction is performed using any wavelength of visible or ultraviolet light. In some embodiments, a crosslinking reaction is completed within 0.1, 0.25, 0.5, 1, 5, or 10 seconds. In some embodiments, a crosslinking reaction is completed within 20, 30, 40, 50, or 60 seconds. In some embodiments, the method comprises irradiating the biological sample with UV light, such as a 350-400 nm wavelength of light, for between 10 seconds and 10 minutes, between 10 seconds and 5 minutes, between 10 seconds and 2 minutes, between 10 seconds and 1 minute, between 30 seconds and 1 minute, or between 30 seconds and 5 minutes. In some embodiments, a crosslinking reaction is completed within 0.5, 1, 5, 10, 20, 30, 40, 50, or 60 minutes. In some embodiments, a crosslinking reaction has negligible effects on bases that neighbor the photoreactive nucleobase. In some embodiments, other photochemical nucleic acid crosslinking agents, including psoralen and coumarin is used in combination with the photoreactive nucleobases disclosed herein.

In some embodiments, the crosslinkable moiety comprises a coumarin and the photoactivation comprises irradiating the biological sample using a 350 nm wavelength of light. In some embodiments, the crosslinkable moiety comprises a psoralen and the photoactivation comprises irradiating the biological sample using a 365 nm wavelength of light. In some embodiments, the crosslinkable moiety comprises a ^{CNV}K or ^{CNV}D and the photoactivation comprises irradiating the biological sample using a 365 nm wavelength of light. In some embodiments, the crosslinkable moiety comprises a ^{PC}X or ^{PCX}D and the photoactivation comprises irradiating the biological sample using a 400 nm wavelength of light. In some embodiments, the crosslinkable moiety comprises a diazirine and the photoactivation comprises irradiating the biological sample using a 365 nm wavelength of light. In some embodiments, the crosslinkable moiety comprises a thiouridine and the photoactivation comprises irradiating the biological sample using a 365 nm wavelength of light.

In some embodiments, a photo-induced crosslink is reversible. In some embodiments, the method comprises reversing the crosslink. In some embodiments, a vinylcarbazole (e.g., ^{CNV}K, ^{CNV}D, ^{PC}X, or ^{PCX}D) crosslink is reversed when exposed to 305 nm UV light. In some embodiments, a vinylcarbazole (e.g., ^{CNV}K, ^{CNV}D, ^{PC}X, or ^{PCX}D) crosslink is reversed when exposed to 312 nm light. In some embodiments, a psoralen crosslink is reversed when exposed to 254 nm light. In some embodiments, a coumarin crosslink is reversed when exposed to 254 nm light.

In some embodiments, the crosslinkable moiety is a photoactivatable nucleotide comprising a coumarin and hybridizes to a thymine (T) base in the complementary strand. In some embodiments, the photoactivatable nucleotide comprises a psoralen and hybridizes to a C, T, or U base in the complementary strand. In some embodiments, the photoactivatable nucleotide comprises a vinylcarbanazole and hybridizes to a C, T, or U base in the complementary strand. In some embodiments, the photoactivatable nucleotide comprises a universal or random base.

In some embodiments, the crosslinkable moiety crosslinks to an adenine (A) nucleobase in the strand of the target nucleic acid hybridized to the oligonucleotide. In some embodiments, the crosslinkable moiety comprises a psoralen capable of crosslinking to an adenine in the hybridized nucleic acid strand. In some embodiments, the crosslinkable moiety comprises a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite). In some embodiments, crosslinkable moiety comprises a 5′-Dimethoxytrityl-5-iodo-2′-deoxyUridine,3′-[(2-cyanoethyl)-(N,N-diisopropyl)]-phosphoramidite (5-I-dU-CE phosphoramidite).

The structure of an exemplary psoralen C2 phosphoramadite crosslinkable moiety is shown below:

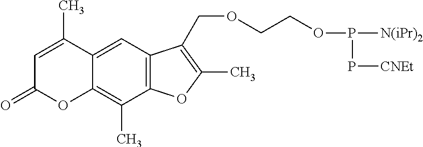

The structure of an exemplary 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite) crosslinkable moiety is shown below:

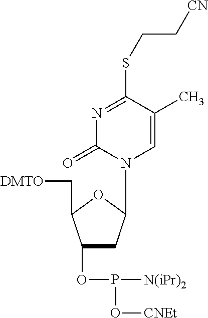

The structure of an exemplary 5′-Dimethoxytrityl-5-iodo-2′-deoxyUridine,3′-[(2-cyanoethyl)-(N,N-diisopropyl)]-phosphoramidite (5-I-dU-CE phosphoramidite) crosslinkable moiety is shown below:

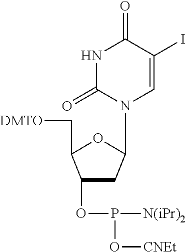

The photoreactive nucleotides may be photo-activated by UV light, such as a 350-400 nm wavelength of light, to photo-activate and crosslink the crosslinkable moiety of the hybridized second nucleic acid strands to the first nucleic acid strand and detectably labeled probes. In some embodiments, the crosslinkable moiety is crosslinked to the complementary strand at a 355 nm wavelength of light. In some embodiments, the purine bases of the target nucleic acid are unreactive to photo-activated crosslinking. In some embodiments, the pyrimidine bases of the complementary strand are reactive to photo-activated crosslinking. In some embodiments, the purine bases of the target nucleic acid are reactive to crosslinking. In some embodiments, the reactivity is to a psoralen, 5-I-dU-CE, 4-Thio-dT-CE, or any other crosslinkable moiety configured to crosslink with nucleobases including adenine.

The photo-activated crosslinking step may be optimized to prevent DNA damage. In some embodiments, the photo-activated crosslinking does not cause significant DNA damage. In some embodiments, the photo-activated crosslinking between the hybridized second nucleic acid strands and the first nucleic acid strand, and/or between the second nucleic acid strands and the detectably labeled probes increases the UV melting temperature of the duplex compared to prior to the crosslinking. In some embodiments, the UV melting temperature is increased by about 30° C. per photoreactive nucleotide in the hybridization region.

FIG. 1A shows the formation of an example crosslink between a circularizable probe and a second nucleic acid strand, wherein the crosslinking occurs before contacting the biological sample (e.g., in vitro). In some cases, 2, 3, 4, 5, or more crosslinks are formed between the circularizable probe and the second nucleic acid strand. In this example, the circularizable probe is a single linear polynucleotide. In some embodiments, the circularizable probe comprises two target-complementary arms that correspond to two separate regions on a target nucleic acid molecule. In some embodiments, the circularizable probe further comprises a sequence complementary to the second nucleic acid strand (e.g., a second nucleic acid strand hybridizing region). The circularizable probe and the second nucleic acid strand are contacted in vitro, wherein the second nucleic acid strand hybridizes to the complementary sequence comprised by the circularizable probe. In some cases, as shown in FIG. 1A, the second nucleic acid strand and circularizable probe are then crosslinked in vitro. In some embodiments, the crosslinking occurs by irradiating the mixture comprising the second nucleic acid strand hybridized to the circularizable probe with UV light prior to contacting the biological sample. In some embodiments, the mixture is irradiated using a 350-400 nm wavelength of light. In some embodiments, the mixture is irradiated using a 360-370 nm wavelength of light. In some embodiments, the mixture is irradiated for from about 1 minute to about 10 minutes.

In some embodiments, the crosslinking occurs after contacting the circularizable probe or probe set to the biological sample. In some embodiments, the crosslinking occurs by irradiating a mixture comprising the second nucleic acid strand and the circularizable probe or probe set with UV light after contacting the biological sample. In some embodiments, the mixture is irradiated using a 350-400 nm wavelength of light. In some embodiments, the mixture is irradiated using a 360-370 nm wavelength of light. In some embodiments, the mixture is irradiated for from about 1 minute to about 10 minutes.

In some embodiments, the second nucleic acid strand is crosslinked to the circularizable probe or probe set prior to contacting of the circularizable probe or probe set to the target nucleic acid. In some embodiments, the second nucleic acid strand is crosslinked to the circularizable probe or probe set that is ligated following hybridization to the target nucleic acid, optionally following splint-mediated ligation of the circularizable probe set. In some embodiments, the second nucleic acid is crosslinked to the circularizable probe or probe set as it is ligated to form the circular nucleic acid template. In some embodiments, the second nucleic acid strand is crosslinked to the circular nucleic acid template.

In some embodiments, the second nucleic acid strand is crosslinked to the circularizable probe or probe set prior after contacting of the circularizable probe or probe set to the target nucleic acid. In some embodiments, the second nucleic acid strand is crosslinked to the circularizable probe or probe set after hybridization to the target nucleic acid and ligation of the circularizable probe or probe set. In some embodiments, the second nucleic acid strand is crosslinked to a circularizable probe set following splint-mediated ligation of the circularizable probe set. In some embodiments, the second nucleic acid is crosslinked to the circularizable probe or probe set after it is ligated to form the circular nucleic acid template. In some embodiments, the second nucleic acid strand is crosslinked to the circular nucleic acid template following formation of the circular nucleic acid template.

FIG. 1B shows an example composition of a circularizable probe or probe set crosslinked to a second nucleic acid strand and contacted with a target nucleic acid within a biological sample. In this example, the circularizable probe or probe set comprises a first hybridization region that complements a first sequence comprised in the target nucleic acid and a second hybridization region that complements a second sequence comprised in the target nucleic acid (e.g., two target-complementary arms that hybridize to the target nucleic acid). The first and second hybridization regions comprised on the circularizable probe or probe set form an optional ligation site in the gap that forms between both regions upon hybridization to the target nucleic acid. The circularizable probe or probe set further comprises an optional barcode sequence. In some embodiments, the circularizable probe set comprises a second optional ligation site comprised within a region of the probe set not hybridized to the target nucleic acid. In this example, the second nucleic acid strand is crosslinked to the circularizable probe or probe set at the site of hybridization of the second nucleic acid strand to the circularizable probe or probe set. In some embodiments, the crosslinking is created using crosslinkable moieties comprised on nucleotides of the second nucleic acid strand. In some embodiments, the crosslinking is created using crosslinkable moieties comprised on nucleotides of the circularizable probe or probe set in the region where the second nucleic acid strand hybridizes to the probe or probe set.

In some embodiments, the method comprises de-crosslinking the circular nucleic acid template from the second nucleic acid strand by irradiating the biological sample to reverse one or more of the interstrand linkages between the circular nucleic acid template and the second nucleic acid strand. In some embodiments, the de-crosslinking comprises irradiating the sample with a wavelength between about 300 nm and about 320 nm. In some embodiments, the de-crosslinking comprises irradiating the sample with a wavelength between about 305 nm and about 312 nm. In some embodiments, the de-crosslinking comprises irradiating the sample with a wavelength between about 308 nm and about 320 nm, about 310 nm and about 320 nm, about 312 nm and about 320 nm, about 314 nm and about 320 nm, about 316 nm and about 320 nm, and about 318 nm and about 320 nm. In some embodiments, the circular nucleic acid template is crosslinked to the second nucleic acid strand via one or more ^{CNV}K interstrand linkages, and the de-crosslinking comprises irradiating the sample with a wavelength between about 305 nm and about 312 nm.

In some embodiments, the de-crosslinking comprises irradiating the sample with a wavelength between about 250 nm and about 300 nm. In some embodiments, the de-crosslinking comprises irradiating the sample with a wavelength of between about 250 nm and about 290 nm, about 250 nm and about 280 nm, about 250 and about 270 nm, and about 250 and about 260 nm. In some embodiments, the de-crosslinking comprises irradiating the sample with a wavelength of about 254 nm. In some embodiments, the circular nucleic acid template is crosslinked to the second nucleic acid strand via one or more psoralen interstrand linkages, and the de-crosslinking comprises irradiating the sample with a wavelength between about 250 nm and about 300 nm. In some embodiments, the circular nucleic acid template is crosslinked to the second nucleic acid strand via one or more psoralen interstrand linkages, and the de-crosslinking comprises irradiating the sample with a wavelength of about 254 nm.

In some embodiments, irradiating the biological sample to reverse one or more of the interstrand linkages comprises irradiating the sample for a specified amount of time. In some embodiments, reversing the one or more interstrand linkages comprises irradiating the biological sample for about 1 second to about 10 minutes. In some embodiments, reversing the one or more interstrand linkages comprises irradiating the biological sample for about 1 second, about 30 seconds, about 1 minute, about 2 minutes, about 3 minutes, about 4 minutes, about 5 minutes, or about 10 minutes, or for a range between any of the aforementioned time periods. In some embodiments, reversing the one or more interstrand linkages comprises irradiating the biological sample for about 3 minutes. In some embodiments, reversing the one or more interstrand linkages comprises irradiating the biological sample for no more than about 1 minute, about 2 minutes, about 3 minutes, about 4 minutes, or about 5 minutes. In some embodiments, the biological sample is irradiated for no more than about 5 minutes. In some embodiments, irradiating the biological sample leads to partial or complete reversal of the interstrand linkages between the circular nucleic acid template and the second nucleic acid strand.

### D. Example Rolling Circle Amplification Workflows Comprising Crosslinking and De-Crosslinking

In some embodiments, disclosed herein is a method for analyzing a biological sample, comprising contacting the biological sample with a circularizable probe, wherein the circularizable probe comprises a first hybridization region (e.g., a 5′ hybridization region) that hybridizes to a first sequence in a target nucleic acid, and a second hybridization region (e.g., a 3′ hybridization region) that hybridizes to a the target nucleic acid, wherein the circularizable probe is crosslinked to a second nucleic acid strand prior to contacting the biological sample (e.g., in vitro). In some embodiments, the method further comprises one or more stringent washes of the circularizable probe within the biological sample, wherein circularizable probes not hybridized to the target nucleic acid are removed. In some embodiments, the method further comprises ligating the first and second hybridization regions of the circularizable probe hybridized to the target nucleic acid molecule to form a circular nucleic acid template, whereas circularizable probes that are not hybridized to the target nucleic acid molecule are not ligated. In some embodiments, circularizable probes or probe sets that are not hybridized to the target nucleic acid molecule are removed through the one or more stringent washing steps prior to ligation. In some embodiments, the method further comprises contacting the circular nucleic acid template with a polymerase, wherein the polymerase binds to the second nucleic acid strand crosslinked to the circular nucleic acid template. In some embodiments, the method further comprises extending the second nucleic acid strand using the polymerase to generate an extended priming sequence using the circular nucleic acid template as a template.

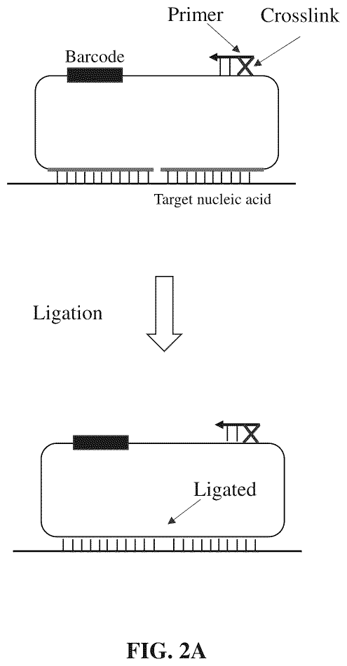

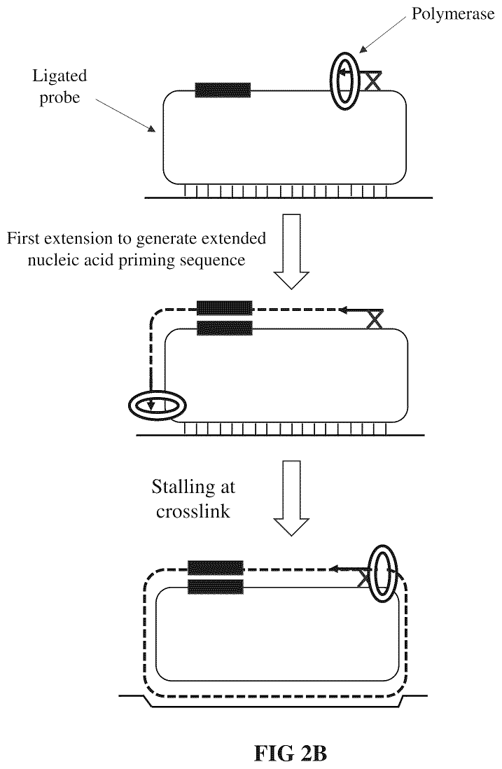

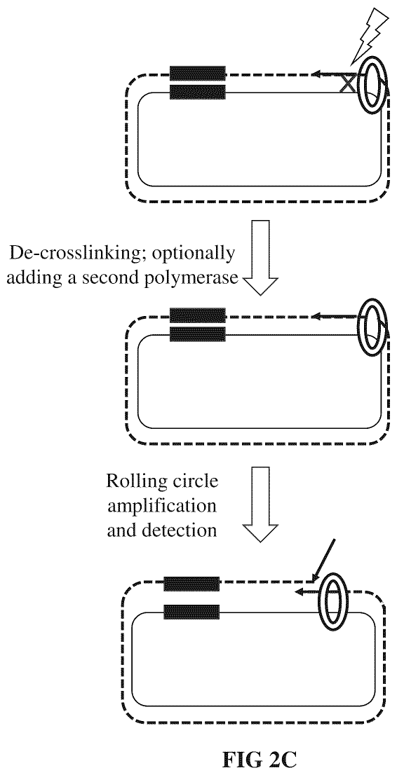

An example workflow is described in FIG. 2A-C. FIG. 2A shows a circularizable probe crosslinked to a second nucleic acid strand at one or more nucleotides. The circularizable probe comprises an optional barcode sequence and two target-complementary arms comprised on both the 5′ and 3′ ends of the linear polynucleotide. The target-complementary arms are hybridized to two complementary sequences on a target nucleic acid, wherein the two complementary sequences are comprised on the target nucleic acid in close proximity to one another. This proximity facilitates ligation (e.g., template-mediated ligation) as shown in FIG. 2A, thereby forming a circular nucleic acid template. The circular nucleic acid template hybridized to the target nucleic acid is contacted with a polymerase (e.g., Phi29 DNA polymerase) as shown in FIG. 2B, wherein the polymerase binds to the second nucleic acid strand hybridized and crosslinked to the circular nucleic acid template. The polymerase then extends the second nucleic acid strand in a first extension step using the circular nucleic acid template as a template, thereby generating an extended priming sequence. The extended priming sequence is a polynucleotide comprising the complement sequence to the circular nucleic acid template, thereby comprising a complement to the barcode sequence of the circular nucleic acid template. While generating the extended priming sequence, the polymerase also displaces the target nucleic acid, thereby disrupting the hybridization between the target nucleic acid and circular nucleic acid template. Upon reaching at least one crosslink between the second nucleic acid strand and the circular nucleic acid template, the polymerase stalls. In some embodiments, the stalled polymerase remains bound to the circular nucleic acid template. In some embodiments, the stalled polymerase falls off the circular nucleic acid template. As shown in FIG. 2C, following stalling of the polymerase, the crosslink between the second nucleic acid strand and the circular nucleic acid template is reversed (e.g., by irradiating the circular nucleic acid template with a wavelength between about 300 nm and about 320 nm for one to 10 minutes). In some embodiments, the circular nucleic acid template is contacted with a second polymerase following de-crosslinking. The method then comprises performing rolling circle amplification by further extending the extended priming sequence with the polymerase, thereby generating an RCA product comprising the complement of the circular nucleic acid template sequence. The method further comprises detecting the RCA product, in some embodiments by detecting the complement of the optional barcode sequence comprised on the RCA product.

In some embodiments, disclosed herein is a method for analyzing a biological sample, comprising contacting the biological sample with a circularizable probe, wherein the circularizable probe comprises a first hybridization region (e.g., a 5′ hybridization region) that hybridizes to a first sequence in a target nucleic acid, and a second hybridization region (e.g., a 3′ hybridization region) that hybridizes to a corresponding second sequence in the target nucleic acid, wherein the circularizable probe is crosslinked to a second nucleic acid strand after contacting the biological sample (e.g., in situ). In some embodiments, the method further comprises one or more stringent washes of the circularizable probe within the biological sample, wherein circularizable probes not hybridized to the target nucleic acid are removed. In some embodiments, the circularizable probe further comprises a second nucleic acid strand hybridizing sequence, and the method further comprises hybridizing a second nucleic acid strand to the circularizable probe hybridized to the target nucleic acid at the second nucleic acid strand hybridizing sequence. In some embodiments, the method further comprises crosslinking the second nucleic acid strand to the circularizable probe at the second nucleic acid strand hybridizing sequence within the biological sample. In some embodiments, the method further comprises ligating the first and second hybridization regions of the circularizable probe hybridized to the target nucleic acid molecule to form a circular nucleic acid template, whereas circularizable probes that are not hybridized to the target nucleic acid molecule are not ligated. In some embodiments, circularizable probes that are not hybridized to the target nucleic acid molecule are removed through the one or more stringent washing steps prior to ligation. In some embodiments, the method further comprises contacting the circular nucleic acid template with a polymerase, wherein the polymerase binds to the second nucleic acid strand crosslinked to the circular nucleic acid template. In some embodiments, the method further comprises extending the second nucleic acid strand using the polymerase to generate an extended priming sequence using the circular nucleic acid template as a template.

In some embodiments, disclosed herein is a method for analyzing a biological sample, comprising contacting the biological sample with a circularizable probe set, wherein the circularizable probe set comprises two nucleic acid probes, wherein a first nucleic acid probe comprises a first hybridization region (e.g., a 5′ hybridization region) that hybridizes to a first sequence in a target nucleic acid, and a second nucleic acid probe comprises a second hybridization region (e.g., a 3′ hybridization region) that hybridizes to a corresponding second sequence in the target nucleic acid, wherein the circularizable probe set is crosslinked to a second nucleic acid strand on either the first or second nucleic acid probe, wherein the crosslinking occurs before contacting the biological sample (e.g., in vitro). In some embodiments, the method further comprises one or more stringent washes of the circularizable probe set within the biological sample, wherein circularizable probe sets not hybridized to the target nucleic acid are removed. In some embodiments, the crosslinking occurs after contacting the biological sample (e.g., in situ). In some embodiments, the first nucleic acid probe further comprises a first splint complementary region (e.g., a 3′ region that hybridizes to a first portion of a splint oligonucleotide), and the second nucleic acid probe further comprises a second splint complementary region (e.g., a 5′ region that hybridizes to a second portion of a splint oligonucleotide). In some embodiments, the circularizable probe set further comprises a second nucleic acid strand hybridizing sequence, wherein the second nucleic acid strand hybridizing sequence is comprised on either the first or the second nucleic acid probe.

In some embodiments, ligation of the circularizable probe set at the hybridization regions hybridized to the target nucleic acid and the splint complementary regions hybridized to the splint oligonucleotide forms a circular nucleic acid template. In some embodiments, the method further comprises contacting the circular nucleic acid template with a polymerase, wherein the polymerase binds to the second nucleic acid strand crosslinked to the circular nucleic acid template. In some embodiments, the method further comprises extending the second nucleic acid strand using the polymerase to generate an extended priming sequence using the circular nucleic acid template as a template.

In some embodiments, disclosed herein is a method for analyzing a biological sample, comprising contacting the biological sample with a circularizable probe set, wherein the circularizable probe set comprises two nucleic acid probes, wherein a first nucleic acid probe comprises a first hybridization region (e.g., a 5′ hybridization region) that hybridizes to a first sequence in a target nucleic acid, and a second nucleic acid probe comprises a second hybridization region (e.g., a 3′ hybridization region) that hybridizes to a corresponding second sequence in the target nucleic acid, wherein the circularizable probe set is crosslinked to a second nucleic acid strand on either the first or second nucleic acid probe, wherein the crosslinking occurs after contacting the biological sample (e.g., in situ). In some embodiments the first nucleic acid probe further comprises a first splint complementary region (e.g., a 3′ region that hybridizes to a first portion of a splint oligonucleotide), and the second nucleic acid probe further comprises a second splint complementary region (e.g., a 5′ region that hybridizes to a second portion of a splint oligonucleotide). In some embodiments, the circularizable probe set further comprises a second nucleic acid strand hybridizing sequence, wherein the second nucleic acid strand hybridizing sequence is comprised on either the first or the second nucleic acid probe. In some embodiments, the method further comprises one or more stringent washes of the circularizable probe set within the biological sample, wherein circularizable probe sets not hybridized to the target nucleic acid are removed. In some embodiments, the method further comprises contacting the circularizable probe set with a splint oligonucleotide, wherein the splint oligonucleotide hybridizes to the first splint complementary region comprised on the first nucleic acid probe, and to the second splint complementary region comprised on the second nucleic acid probe. In some embodiments, the method further comprises ligating the first and second hybridization regions of the circularizable probe set hybridized to the target nucleic acid molecule, whereas circularizable probe sets that are not hybridized to the target nucleic acid molecule are not ligated (e.g., removed through the one or more stringent washing steps prior to ligation). In some embodiments, the method further comprises ligating the first nucleic acid probe and the second nucleic acid probe of the circularizable probe set hybridized to the splint oligonucleotide. In some embodiments, ligation of the circularizable probe set at the hybridization regions hybridized to the target nucleic acid and the splint complementary regions hybridized to the splint oligonucleotide forms a circular nucleic acid template. In some embodiments, the method further comprises hybridizing a second nucleic acid strand at the second nucleic acid strand hybridizing sequence comprised on the circular nucleic acid template hybridized to the target nucleic acid. In some embodiments, the method further comprises crosslinking the second nucleic acid strand to the circular nucleic acid template at the second nucleic acid strand hybridizing sequence within the biological sample. In some embodiments, the method further comprises contacting the circular nucleic acid template with a polymerase, wherein the polymerase binds to the second nucleic acid strand crosslinked to the circular nucleic acid template. In some embodiments, the method further comprises extending the second nucleic acid strand using the polymerase to generate an extended priming sequence using the circular nucleic acid template as a template.

In some embodiments, disclosed herein is a method for analyzing a biological sample, comprising contacting the biological sample with a circularizable probe, wherein the circularizable probe comprises a first hybridization region (e.g., a 5′ hybridization region) that hybridizes to a first sequence in a target nucleic acid, and a second hybridization region (e.g., a 3′ hybridization region) that hybridizes to a corresponding second sequence in the target nucleic acid, wherein the circularizable probe is crosslinked to a second nucleic acid strand prior to contacting the biological sample (e.g., in vitro). In some embodiments, the circularizable probe further comprises a separate primer binding sequence that is complementary to a primer. In some embodiments, the method further comprises one or more stringent washes of the circularizable probe within the biological sample, wherein circularizable probes not hybridized to the target nucleic acid are removed. In some embodiments, the method further comprises ligating the first and second hybridization regions of the circularizable probe hybridized to the target nucleic acid molecule to form a circular nucleic acid template, whereas circularizable probes that are not hybridized to the target nucleic acid molecule are not ligated (e.g., removed through the one or more stringent washing steps prior to ligation). In some embodiments, the method further comprises contacting the circular nucleic acid with a primer that hybridizes to the separate primer binding sequence. In some embodiments, the method further comprises contacting the circular nucleic acid template with a polymerase, wherein the polymerase binds to the primer hybridized to the circular nucleic acid template. In some embodiments, the method further comprises extending the primer using the polymerase to generate an extended priming sequence using the circular nucleic acid template as a template.

In some embodiments, disclosed herein is a method for analyzing a biological sample, comprising contacting the biological sample with a circularizable probe, wherein the circularizable probe comprises a first hybridization region (e.g., a 5′ hybridization region) that hybridizes to a first sequence in a target nucleic acid, and a second hybridization region (e.g., a 3′ hybridization region) that hybridizes to a corresponding second sequence in the target nucleic acid, wherein the circularizable probe is crosslinked to a second nucleic acid strand prior to contacting the biological sample (e.g., in vitro). In some embodiments, the method further comprises one or more stringent washes of the circularizable probe within the biological sample, wherein circularizable probes not hybridized to the target nucleic acid are removed. In some embodiments, the method further comprises ligating the first and second hybridization regions of the circularizable probe hybridized to the target nucleic acid molecule to form a circular nucleic acid template, whereas circularizable probes that are not hybridized to the target nucleic acid molecule are not ligated (e.g., removed through the one or more stringent washing steps prior to ligation). In some embodiments, the method further comprises contacting the target nucleic acid hybridized to the circular nucleic acid template with an endonuclease, wherein the endonuclease digests the target nucleic acid hybridized to the circular nucleic acid template (e.g., wherein the target nucleic acid is an RNA, the circular nucleic acid template is DNA, and the endonuclease is RNaseH, wherein RNaseH digests RNA hybridized to DNA). In some embodiments, the endonuclease forms one or more priming sequences from the target nucleic acid hybridized to the circular nucleic acid template, wherein the priming sequences remain hybridized to the circular nucleic acid template. In some embodiments, digestion and/or cleavage by the endonuclease forms a free 3′ end of the target nucleic acid. In some embodiments, the method further comprises contacting the circular nucleic acid template with a polymerase, wherein the polymerase binds to the one or more priming sequences hybridized to the circular nucleic acid template. In some embodiments, the method further comprises extending the one or more priming sequences using the polymerase to generate an extended priming sequence using the circular nucleic acid template as a template.

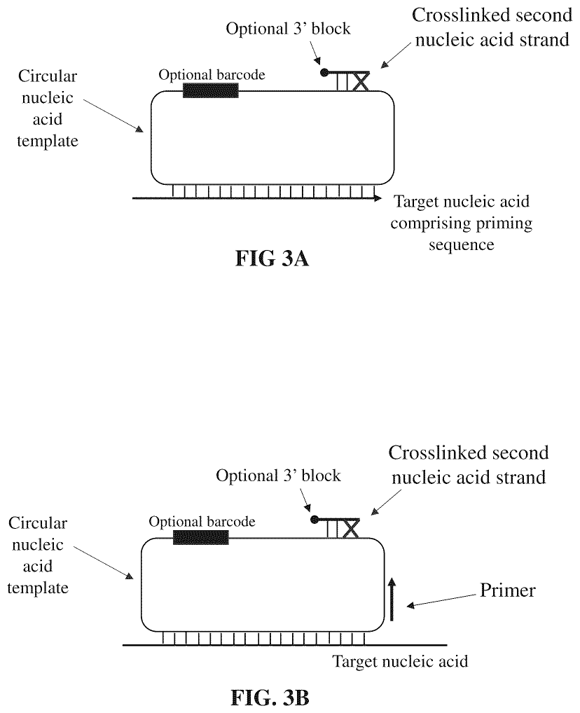

In some embodiments, disclosed herein is a method for analyzing a biological sample, comprising contacting the biological sample with a circularizable probe, wherein the circularizable probe comprises a first hybridization region (e.g., a 5′ hybridization region) that hybridizes to a first sequence in a target nucleic acid, and a second hybridization region (e.g., a 3′ hybridization region) that hybridizes to a corresponding second sequence in the target nucleic acid, wherein the circularizable probe is crosslinked to a second nucleic acid strand prior to contacting the biological sample (e.g., in vitro). In some embodiments, the second nucleic acid strand comprises a 3′ block, wherein the 3′ block prevents extension of the second nucleic acid by a polymerase. In some embodiments, the 3′ block is a dideoxynucleotide comprised on the second nucleic acid strand. In some embodiments, the method further comprises one or more stringent washes of the circularizable probe within the biological sample, wherein circularizable probes not hybridized to the target nucleic acid are removed. In some embodiments, the method further comprises ligating the first and second hybridization regions of the circularizable probe hybridized to the target nucleic acid molecule to form a circular nucleic acid template, whereas circularizable probes that are not hybridized to the target nucleic acid molecule are not ligated (e.g., removed through the one or more stringent washing steps prior to ligation). FIG. 3A shows an example wherein the circular nucleic acid template is crosslinked to the 3′ blocked second nucleic acid strand and hybridized to the target nucleic acid comprising one or more priming sequences. In some embodiments, the method further comprises contacting the target nucleic acid hybridized to the circular nucleic acid template with an endonuclease, wherein the endonuclease digests the target nucleic acid hybridized to the circular nucleic acid template (e.g., wherein the target nucleic acid is an RNA, the circular nucleic acid template is DNA, and the endonuclease is RNaseH, wherein RNaseH digests RNA hybridized to DNA). In some embodiments, the endonuclease forms one or more priming sequences from the target nucleic acid hybridized to the circular nucleic acid template, wherein the priming sequences remain hybridized to the circular nucleic acid template. In some embodiments, digestion and/or cleavage by the endonuclease forms a free 3′ end of the target nucleic acid. In some embodiments, the method further comprises contacting the circular nucleic acid template with a polymerase, wherein the polymerase binds to the one or more priming sequences hybridized to the circular nucleic acid template. In some embodiments, the method further comprises extending the one or more priming sequences using the polymerase to generate an extended priming sequence using the circular nucleic acid template as a template. In some embodiments, the second nucleic acid strand is not extended by the polymerase due to the 3′ block comprised on the second nucleic acid strand, thereby preventing off-target formation of the extended priming sequence.

In some embodiments, disclosed herein is a method for analyzing a biological sample, comprising contacting the biological sample with a circularizable probe, wherein the circularizable probe comprises a first hybridization region (e.g., a 5′ hybridization region) that hybridizes to a first sequence in a target nucleic acid, and a second hybridization region (e.g., a 3′ hybridization region) that hybridizes to a corresponding second sequence in the target nucleic acid, wherein the circularizable probe is crosslinked to a second nucleic acid strand prior to contacting the biological sample (e.g., in vitro). In some embodiments, the second nucleic acid strand comprises a 3′ block, wherein the 3′ block prevents extension of the second nucleic acid by a polymerase. In some embodiments, the 3′ block is a dideoxynucleotide comprised on the second nucleic acid strand. In some embodiments, the circularizable probe further comprises a separate primer binding sequence that is complementary to a primer. In some embodiments, the method further comprises one or more stringent washes of the circularizable probe within the biological sample, wherein circularizable probes not hybridized to the target nucleic acid are removed. In some embodiments, the method further comprises ligating the first and second hybridization regions of the circularizable probe hybridized to the target nucleic acid molecule to form a circular nucleic acid template, whereas circularizable probes that are not hybridized to the target nucleic acid molecule are not ligated (e.g., removed through the one or more stringent washing steps prior to ligation). In some embodiments, the method further comprises contacting the circular nucleic acid with a primer that hybridizes to the separate primer binding sequence. FIG. 3B shows an example composition resulting from this method, wherein the circular nucleic acid template is crosslinked to the 3′ blocked second nucleic acid strand and hybridized to both the primer and the target nucleic acid.

In some embodiments, the method further comprises contacting the circular nucleic acid template with a polymerase, wherein the polymerase binds to the primer hybridized to the circular nucleic acid template. In some embodiments, the method further comprises extending the primer using the polymerase to generate an extended priming sequence using the circular nucleic acid template as a template. In some embodiments, the second nucleic acid strand is not extended by the polymerase due to the 3′ block comprised on the second nucleic acid strand, thereby preventing off-target formation of the extended priming sequence.

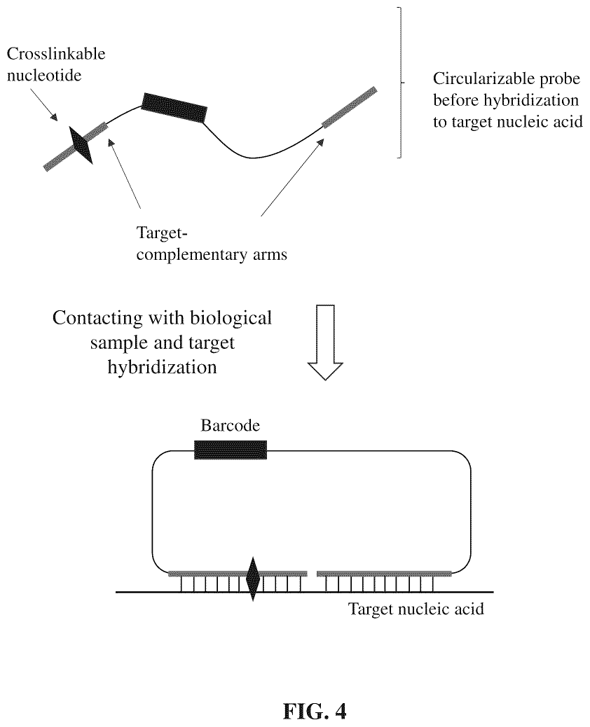

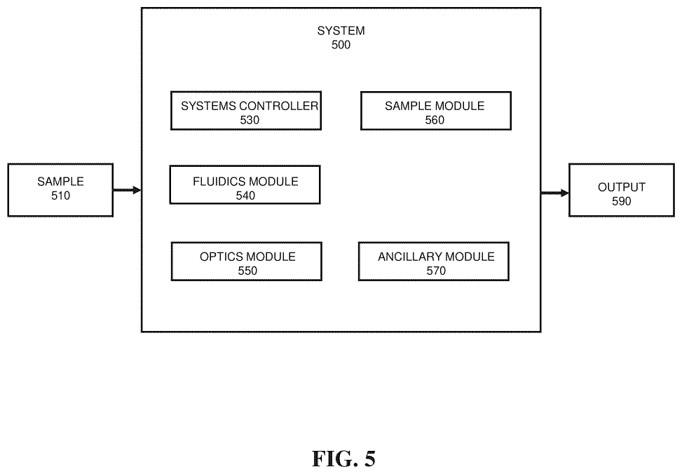

In some embodiments, disclosed herein is a method for analyzing a biological sample, comprising contacting the biological sample with a circularizable probe, wherein the circularizable probe comprises a first hybridization region (e.g., a 5′ hybridization region) that hybridizes to a first sequence in a target nucleic acid, and a second hybridization region (e.g., a 3′ hybridization region) that hybridizes to a corresponding second sequence in the target nucleic acid, wherein the circularizable probe comprises one or more crosslinkable bases within the first and/or second hybridization regions. FIG. 4 shows an example composition resulting from this method, wherein a circularizable probe comprising one or more crosslinkable nucleotides within one of the two target-complementary arms is hybridized to a target nucleic acid within the biological sample. In some embodiments, the method further comprises crosslinking the circular nucleic acid to the target nucleic acid, wherein the crosslinking comprises irradiating the biological sample. In some embodiments, the method further comprises contacting the circular nucleic acid template with a polymerase to extend the one or more priming sequences using the polymerase to generate an extended priming sequence using the circular nucleic acid template as a template. In some embodiments, the polymerase stalls during or after generation of the extended priming sequence upon reaching the one or more crosslinks between the second nucleic acid strand and the circular nucleic acid template.

In some embodiments, disclosed herein is a method for analyzing a biological sample, comprising contacting the biological sample with a circularizable probe set, wherein the circularizable probe set comprises two nucleic acid probes, and one or both of the nucleic acid probes is crosslinked to a second nucleic acid strand. In some embodiments, one or both of the nucleic acid probes is crosslinked to a second nucleic acid strand before contacting the biological sample. In some embodiments, the second nucleic acid strand comprises a 3′ block, wherein the 3′ block prevents extension of the second nucleic acid by a polymerase. In some embodiments, the 3′ block is a dideoxynucleotide comprised by the second nucleic acid strand. After contacting the biological sample, the circularizable probe set is circularized according to any of the methods disclosed herein to generate a circular nucleic acid template, and a priming sequence hybridized to the circular nucleic acid template is extended using a polymerase. In some embodiments, additional processes are performed as described elsewhere in the present application. In some embodiments, these processes include de-crosslinking, contacting the sample with another polymerase, rolling circle amplification, and/or detection. In some embodiments, a separate primer comprising the priming sequence is contacted with the biological sample. In some embodiments, the target nucleic acid comprises the priming sequence.

In some embodiments, disclosed herein is a method for analyzing a biological sample, comprising contacting the biological sample with a plurality of circularizable probes, wherein each circularizable probe comprises a hybridization region complementary to a different target nucleic acid sequence, and wherein at least a subset of the circularizable probes are crosslinked to primers before contacting the biological sample. In some embodiments, the method further comprises allowing the circularizable probes to hybridize to their respective target nucleic acid sequences present in the biological sample. In some embodiments, the method further comprises performing one or more stringent washes of the biological sample after allowing the circularizable probes to hybridize to their respective target nucleic acid sequences present in the biological sample. In some embodiments, the method further comprises ligating the hybridized circularizable probes to generate a plurality of circular nucleic acid templates. In some embodiments, the method further comprises extending the primers using a polymerase to generate extended priming sequences using the circular nucleic acid templates as templates. In some embodiments, the method further comprises de-crosslinking the circular nucleic acid templates from the primers. In some embodiments, the method further comprises extending the extended priming sequences to generate a plurality of rolling circle amplification products (RCPs) in the biological sample.

In some embodiments, the polymerase stalls during or after generation of the extended priming sequence (e.g., upon reaching the one or more crosslinks between the second nucleic acid strand and the circular nucleic acid template). In some embodiments, the stalled polymerase remains bound to the circular nucleic acid template. In some embodiments, the stalled polymerase falls off the circular nucleic acid template. In some embodiments, the method further comprises de-crosslinking the circular nucleic acid templates from the second nucleic acid strands (e.g., by irradiating the circular nucleic acid template with a wavelength between about 300 nm and about 320 nm for about one to 10 minutes). In some embodiments, the method further comprises contacting the biological sample with a second polymerase, wherein the second polymerase binds to the extended priming sequence and the circular nucleic acid template. In some embodiments, the second polymerase is additional molecules of the same type of polymerase used to extend the nucleic acid priming sequence prior to the polymerase stalling. In some embodiments, the method further comprises performing rolling circle amplification (RCA) by further extending the extended priming sequence using the circular nucleic acid template as a template, thereby forming an RCA product. In some embodiments, the method further comprises detecting the RCA product within the biological sample. In some embodiments, a signal associated with the RCA product is detected in situ at the location of the target nucleic acid associated with the RCA product within the biological sample.

### III. Detection and Analysis

In some aspects, the provided methods involve analyzing, e.g., detecting or determining, one or more sequences present in the rolling circle amplification product generated from the circular nucleic acid template. In some embodiments, the detecting is performed at one or more locations in the biological sample. In some embodiments, the locations are the locations of RNA transcripts in the biological sample. In some embodiments, the locations are the locations at which the probes or probe sets hybridize to the RNA transcripts in the biological sample. In some embodiments, the probes or probe sets are ligated and amplified by rolling circle amplification.

In some embodiments, detecting the one or more sequences present in the probes or probe sets in the biological sample is performed. In some embodiments, the one or more sequences are one or more barcode sequences or complements thereof.

In some embodiments, the detecting comprises a plurality of repeated cycles of hybridization and removal of probes (e.g., detectably labeled probes, or intermediate probes that bind to detectably labeled probes) to the primary probe or probe set hybridized to the target nucleic acid, or to a rolling circle amplification product generated from the probe or probe set hybridized to the target nucleic acid. In some embodiments, the primary probe or probe set is a circularizable probe or circularizable probe set.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization assays, or sequencing by hybridization).

In some embodiments, the detecting comprises binding an intermediate probe directly or indirectly to the primary probe or probe set, binding a detectably labeled probe directly or indirectly to a detection region of the intermediate probe, and detecting a signal associated with the detectably labeled probe. In some embodiments, the method comprises detecting a rolling circle amplification product (RCP) generated using a circular nucleic acid template (e.g., as described in Section II). In some embodiments, detecting the RCP comprises binding an intermediate probe directly or indirectly to the RCP, binding a detectably labeled probe directly or indirectly to a detection region of the intermediate probe, and detecting a signal associated with the detectably labeled probe. In some embodiments, the method comprises performing one or more wash steps to remove unbound and/or nonspecifically bound intermediate probe molecules from the RCP.

In some embodiments, the detecting comprises: detecting signals associated with detectably labeled probes that are hybridized to barcode regions or complements thereof in the RCP; and/or detecting signals associated with detectably labeled probes that are hybridized to intermediate probes which are in turn hybridized to the barcode regions or complements thereof. In some embodiments, the detectably labeled probes are fluorescently labeled.

In some embodiments, the methods comprise detecting the sequence in all or a portion of an RCP, or detecting a sequence of the RCP, such as one or more barcode sequences present in the RCP. In some embodiments, the sequence of the RCP, or barcode thereof, is indicative of a sequence of the target nucleic acid to which the circular nucleic acid template is bound. In some embodiments, the analysis and/or sequence determination comprises detecting a sequence in all or a portion of the RCP. In some embodiments, the detection step involves sequencing by hybridization, sequencing by ligation, sequencing by synthesis, sequencing by binding, sequencing by avidity, and/or fluorescent in situ sequencing (FISSEQ), and/or hybridization-based in situ sequencing. In some embodiments, the detection step is by sequential fluorescent in situ hybridization (e.g., for combinatorial decoding of the barcode sequence or complement thereof).

In some embodiments, the detection or determination comprises hybridizing to the probe directly or indirectly a detection oligonucleotide labeled with a fluorophore, an isotope, a mass tag, or a combination thereof. In some embodiments, the detection or determination comprises imaging one or more detectably labeled probes hybridized to the RCP, directly or indirectly. In some embodiments, the target nucleic acid is an mRNA in a tissue sample, and the detection or determination is performed when the target nucleic acid and/or the amplification product is in situ in the tissue sample.

In some aspects, the provided methods comprise imaging a detectably labeled probe bound directly or indirectly to the RCP and detecting the detectable label. In some embodiments, the detectably labeled probe comprises a detectable label that can be measured and quantitated. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature- oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the assay comprises in situ sequencing of the generated RCPs. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid.

In some embodiments, analyzing, e.g., detecting or determining, one or more sequences present in the biological sample is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (e.g. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes comprise fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In some embodiments, the detecting comprises contacting the biological sample with one or more detectably labeled probes that directly or indirectly hybridize to the barcode sequences or complements thereof (e.g., in RCPs), and dehybridizing the one or more detectably labeled probes. In some embodiments, the contacting and dehybridizing is repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In some embodiments, the detecting step comprises contacting the biological sample with one or more first detectably labeled probes that directly hybridize to the RCPs directly or indirectly via an intermediate probe. In some instances, the detecting step can comprise contacting the biological sample with one or more first detectably labeled probes that indirectly hybridize to the RCPs. In some embodiments, the detecting step comprises contacting the biological sample with one or more first detectably labeled probes that directly or indirectly hybridize to the RCPs.

In some embodiments, the detecting step comprises contacting the biological sample with one or more intermediate probes that directly or indirectly hybridize to the barcode sequences or complements thereof (e.g., of RCPs generated using the circular nucleic acid template), wherein the one or more intermediate probes are detectable using one or more detectably labeled probes. In some embodiments, the detecting step further comprises dehybridizing the one or more intermediate probes and/or the one or more detectably labeled probes from the barcode sequences or complements thereof. In some embodiments, the contacting and dehybridizing are repeated with the one or more intermediate probes, the one or more detectably labeled probes, one or more other intermediate probes, and/or one or more other detectably labeled probes. In some cases, the repeated contacting, detection and dehybridizing allows detection of barcode sequences or complements thereof and identification of the corresponding sequences of signal codes (e.g., fluorophore sequences assigned to the corresponding barcode sequences or complements thereof).

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. Nos. 5,599,675; 5,750,341; 6,969,488; 6,172,218; and 6,306,597, all of which are herein incorporated by reference in their entireties.

In some embodiments, real-time monitoring of DNA polymerase activity is used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some embodiments, the analysis and/or sequence determination involves washing to remove unbound polynucleotides, thereafter revealing a fluorescent product for imaging.

### IV. Samples, Analytes, and Target Sequences

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprises a cell block or a section of a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent processes in the analytical method. In some embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) processes. In some embodiments, one or more post-fixing process is performed after contacting a sample with a polynucleotide disclosed herein, e.g., one or more probes such as a circular or circularizable probe or probe set (e.g., a padlock probe). In some embodiments, one or more post-fixing process is performed after a hybridization complex comprising a probe and a target is formed in a sample. In some embodiments, one or more post-fixing process is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample is embedded in a matrix. In some embodiments, the matrix is a hydrogel matrix. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) are embedded in a 3D matrix. In some embodiments, a 3D matrix comprises a network of natural molecules and/or synthetic molecules that are chemically and/or enzymatically linked, e.g., by crosslinking. In some embodiments, a 3D matrix comprises a synthetic polymer. In some embodiments, a 3D matrix comprises a hydrogel.

In some aspects, a biological sample is embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix. In some embodiments, the matrix is a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof is modified to contain functional groups that can be used as an anchoring site to attach the polynucleotide probes and/or amplification product to a polymer matrix. In some embodiments, a modified probe comprising oligo dT is used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel includes hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl(GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347 (6221): 543-548, 2015, the entire contents of which are incorporated herein by reference.

In some embodiments, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347 (6221): 543-548, 2015 and U.S. Pat. No. 10,059,990, all of which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. In some embodiments, a sample is stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof (e.g, Dil, DiO, DiR, DiD). Other cell membrane stains may include FM and RH dyes or immunohistochemical reagents specific for cell membrane proteins. In some examples, the stain includes but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample is stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### B. Analytes

A biological sample may comprise one or a plurality of analytes of interest. Methods for performing multiplexed assays to analyze two or more different analytes in a single biological sample are provided. The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

In some cases, a ligation product of an endogenous analyte and/or a labeling agent is analyzed. In some embodiments, the ligation product is formed between two or more endogenous analytes. In some embodiments, the ligation product is formed between two or more labeling agents. In some embodiments, the ligation product is an intramolecular ligation of an endogenous analyte. In some embodiments, the ligation product is an intramolecular ligation product or an intermolecular ligation product. In some embodiments, the ligation product is generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. In some embodiments, the RNA comprises circular RNA. In some embodiments, the RNA is a bacterial rRNA (e.g., 16s rRNA or 23s rRNA).

In some embodiments, an analyte is a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes in a sample using one or more labeling agents. In some embodiments, the endogenous analyte is RNA, ssDNA, cell surface or intracellular proteins, or metabolites. In some embodiments, an analyte labeling agent may include an agent that interacts with an analyte (e.g., an endogenous analyte in a sample). In some embodiments, the labeling agents comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds is also identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) processes after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety includes any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, all of which are herein incorporated by reference in their entireties.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent comprises a reporter oligonucleotide and a label. A label can be fluorophore, a radioisotope, a molecule capable of a colorimetric reaction, a magnetic particle, or any other suitable molecule or compound capable of detection. In some embodiments, the label is conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety (ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### V. Compositions, Kits, and Systems

In some aspects, provided herein are compositions comprising any of the crosslinkable probes or probe sets described herein. Also provided herein are kits or systems, for analyzing an analyte in a biological sample according to any of the methods described herein. In some embodiments, provided herein is a kit or system comprising a circular nucleic acid probe (e.g., a circular probe) and a second nucleic acid strand, wherein a first nucleic acid strand comprises the circular probe, wherein the second nucleic acid strand is crosslinked to the circular probe. In some embodiments, the kit or system comprises a circularizable probe or probe set. In some embodiments, provided herein is a kit or system comprising a circularizable probe or circularizable probe set and a second nucleic acid strand, wherein a first nucleic acid strand comprises the circularizable probe, wherein a set of first nucleic acid strands comprises the circularizable probe set, wherein the second nucleic acid strand is crosslinked to the circularizable probe or the circularizable probe set. In some embodiments, provided herein is a kit or system comprising a circular probe or a circularizable probe or a circularizable probe set, wherein a first nucleic acid strand comprises the circular probe or the circularizable probe, or a set of first nucleic acid strands comprises the circularizable probe set, and the circularizable probe or the circularizable probe set is capable of being circularized upon hybridization to a target nucleic acid, and a second nucleic acid strand comprising a sequence complementary to a sequence of the circular probe, the circularizable probe, or the circularizable probe set, wherein the sequence complementary to the sequence of the circular probe or the circularizable probe or circularizable probe set comprises a crosslinkable moiety.

In some embodiments, provided herein is a kit or system comprising a plurality of probes or probe sets for targeting a panel of target nucleic acids (e.g., a panel of different RNAs) wherein each probe or probe set is crosslinked to a second nucleic acid strand. In some embodiments, the sequence of the second nucleic acid strand is common among a plurality of different probes or probe sets.

In some embodiments, provided herein is a kit or system comprising a circularizable probe or a circularizable probe set comprising one or more nucleotides comprising crosslinkable moieties. In some embodiments, a first nucleic acid strand comprises the circularizable probe or a set of first nucleic acid strands comprises the circularizable probe set. In some embodiments, provided herein is a kit or system comprising a circularizable probe or probe set and a second nucleic acid strand comprising a hybridization sequence complementary to a hybridization sequence of the circularizable probe or probe set, wherein a first nucleic acid strand comprises the circularizable probe or a set of first nucleic acid strands comprises the circularizable probe set, wherein the hybridization sequence of the second nucleic acid strand comprises one or more nucleotides comprising crosslinkable moieties. In some embodiments, provided herein is a kit or system comprising a circular probe and a second nucleic acid strand comprising a hybridization sequence complementary to a hybridization sequence of the circular probe, wherein a first nucleic acid strand comprises the circular probe, wherein the hybridization sequence of the second nucleic acid strand comprises one or more nucleotides comprising crosslinkable moieties.

The various components of the kit or system may be present in separate containers or certain compatible components may be pre-combined into a single container. In some embodiments, the kits or systems further contain instructions for using the components of the kit or system to practice the provided methods.

In some embodiments, the kits or systems can contain reagents and/or consumables required for performing one or more processes of the provided methods. In some embodiments, the kits or systems comprise reagents for fixing, embedding, and/or permeabilizing the biological sample. In some embodiments, the kits or systems comprise reagents, such as enzymes and buffers for ligation and/or amplification, such as ligases and/or polymerases. In some embodiments, the kit or system also comprises any of the reagents described herein, e.g., wash buffer and ligation buffer. In some embodiments, the kits or systems comprise reagents for detection and/or sequencing, such as barcode detection probes or detectable labels. In some embodiments, the kits or systems comprise other components, for example nucleic acid primers, enzymes and reagents, buffers, nucleotides, modified nucleotides, reagents for additional assays.

Provided herein are kits or systems, for example comprising one or more polynucleotides (e.g., any of the probes, labeling agents, and/or second nucleic acid strands described herein) and reagents for performing the methods provided herein, for example reagents required for one or more processes comprising hybridization, ligation, amplification, detection, sequencing, and/or sample preparation as described herein. In some embodiments, the kit or system further comprises a target nucleic acid. In some embodiments, any or all of the polynucleotides are DNA molecules. In some embodiments, the target nucleic acid is a messenger RNA molecule. In some embodiments, the kit or system further comprises a ligase, for instance for forming a circular nucleic acid template from the circularizable probe or probe set. In some embodiments, the ligase has DNA-splinted DNA ligase activity. In some embodiments, the kit or system further comprises a splint oligonucleotide, for instance as a template ligation of the circularizable probe set at a second location not hybridized to the target nucleic acid. In some embodiments, the kit or system further comprises a polymerase, for instance for performing amplification of the circular nucleic acid template (e.g., the ligated circularizable probe or probe set). In some embodiments, the polymerase is capable of using the circular nucleic acid template as a template for amplification. In some embodiments, the kit or system further comprises a primer for amplification.

In some aspects, disclosed herein is a kit or system for analyzing a biological sample comprising a circularizable probe or circularizable probe set and a second nucleic acid strand. In some embodiments, a first nucleic acid strand comprises the circularizable probe or a set of first nucleic acid strands comprises the circularizable probe set. In some embodiments, the second nucleic acid strand comprises one or more crosslinkable bases (e.g., at the 5′ nucleotide of the second nucleic acid strand), wherein the second nucleic acid strand is crosslinked to the circularizable probe or circularizable probe set at the one or more crosslinkable bases. In some embodiments, the circularizable probe or circularizable probe set comprise target-complementary arms that are complementary to adjacent sequences comprised on a target nucleic acid. In some embodiments, the circularizable probe or circularizable probe set further comprises a barcode sequence corresponding to the target nucleic acid. In some embodiments, the kit or system further comprises a ligase, for instance for forming a circular nucleic acid template from the circularizable probe or circularizable probe set. In some embodiments, the ligase has DNA-splinted DNA ligase activity. In some embodiments, the kit or system further comprises a splint oligonucleotide, for instance as a template ligation of the circularizable probe set at a second location not hybridized to the target nucleic acid. In some embodiments, the kit or system further comprises a polymerase, for instance for performing amplification of the circular nucleic acid template (e.g., the ligated circularizable probe or circularizable probe set). In some embodiments, the polymerase is capable of using the circular nucleic acid template as a template for amplification. In some embodiments, the kit or system further comprises a primer for amplification. In some embodiments, the kit or system further comprises one or more detectably-labeled probes (e.g., one or more detectable probes) that directly or indirectly hybridize to the barcode sequences or complements thereof (e.g., comprised on the product of the amplification of the circular nucleic acid template). In some embodiments, the kit or system further comprises one or more detectable sequences that directly or indirectly hybridize to the barcode sequences or complements thereof. In some embodiments, the kit or system further comprises reagents required for the detection of the one or more detectable sequences directly or indirectly hybridized to the barcode sequences or complements thereof (e.g., reagents for performing sequencing-by-binding, sequencing-by-synthesis, sequencing-by-hybridization, or sequencing-by-avidity).

In some aspects, disclosed herein is a kit or system for analyzing a biological sample comprising a circularizable probe or circularizable probe set and a second nucleic acid strand. Provided herein is a system comprising a circularizable probe and a second nucleic acid strand, wherein the circularizable probe comprises a hybridization region configured to hybridize to a target nucleic acid, and wherein the second nucleic acid strand comprises a sequence complementary to a sequence of the circularizable probe, and wherein the sequence complementary to the sequence of the circularizable probe comprises a crosslinkable moiety. In some embodiments, the circularizable probe is a padlock probe.

In some embodiments, the second nucleic acid strand comprises one or more crosslinkable bases (e.g., at the 5′ nucleotide of the second nucleic acid strand), wherein the second nucleic acid strand is crosslinked to the circularizable probe or circularizable probe set at the one or more crosslinkable bases. In some embodiments, the circularizable probe or circularizable probe set comprise target-complementary arms that are complementary to adjacent sequences comprised on a target nucleic acid. In some embodiments, the circularizable probe or circularizable probe set further comprises a barcode sequence corresponding to the target nucleic acid. In some embodiments, the kit or system further comprises one or more intermediate probes that directly or indirectly hybridize to the barcode sequences or complements thereof; and/or one or more detectably-labeled probes (e.g., one or more detectable probes) that directly or indirectly hybridize to the one or more intermediate probes. In some embodiments, the kit or system further comprised one or more detectably labeled probes that directly or indirectly bind to the one or more intermediate probes. In some embodiments, the kit or system further comprises reagents required for the detection of the one or more detectable sequences directly or indirectly hybridized to the one or more intermediate probes (e.g., reagents for performing sequencing-by-binding, sequencing-by-synthesis, sequencing-by-hybridization, or sequencing-by-avidity). In some embodiments, the kit or system further comprises reagents for performing sequencing-by-binding, sequencing-by-synthesis, sequencing-by-hybridization, or sequencing-by-avidity of the one or more sequences present in the rolling circle amplification product generated from the circular nucleic acid template.

In some embodiments, the system comprises a optical detection system (e.g., as described in Section VI) configured to detect the one or more sequences present in the rolling circle amplification product generated from the circular nucleic acid template. In some embodiments, the system comprises a light source configured to activate the crosslinkable moiety. In some embodiments, the system comprises a UV light source.

The various components of the kit may be present in separate containers or certain compatible components may be pre-combined into a single container. In some embodiments, the kits further contain instructions for using the components of the kit to practice the provided methods.

In some embodiments, the kits or systems comprise reagents and/or consumables required for performing one or more processes of the provided methods. In some embodiments, the kits or systems comprise reagents for fixing, embedding, and/or permeabilizing the biological sample. In some embodiments, the kits or systems comprise reagents, such as enzymes and buffers for ligation and/or amplification, such as ligases and/or polymerases. In some aspects, the kit or system also comprises any of the reagents described herein, e.g., wash buffer and ligation buffer. In some embodiments, the kits or systems comprises reagents for detection and/or sequencing, such as barcode detection probes or detectable labels. In some embodiments, the kits or systems comprise other components, for example nucleic acid primers, enzymes and reagents, buffers, nucleotides, modified nucleotides, reagents for additional assays.

In some embodiments, disclosed herein is a composition that comprises a mixture containing a circularizable probe or circularizable probe set and a second nucleic acid strand. In some embodiments, the mixture is formed outside of a biological sample (e.g., in vitro). In some embodiments, the mixture comprises the second nucleic acid strand hybridized to the circularizable probe or circularizable probe set. In some embodiments, the mixture comprises the second nucleic acid strand crosslinked to the circularizable probe or circularizable probe set. In some embodiments, the mixture comprises a circular nucleic acid template. In some embodiments, disclosed herein is a composition that comprises a mixture comprising the second nucleic acid strand and the circular nucleic acid template or the circularizable probe or circularizable probe set. FIG. 1A shows example embodiments of these compositions, wherein the mixture comprises the circularizable probe and the second nucleic acid strand hybridized to one another and crosslinked to one another in vitro.

In some embodiments, disclosed herein is a composition that comprises a complex containing a target nucleic acid, a circularizable probe or circularizable probe set (e.g., any of the circularizable probes or circularizable probe sets comprised herein), and a second nucleic acid strand. In some embodiments, the second nucleic acid strand is crosslinked to the circularizable probe or circularizable probe set. In some embodiments, the circularizable probe or circularizable probe set is hybridized to the target nucleic acid.

FIG. 1B shows an example composition comprising a circularizable probe or circularizable probe set crosslinked to a second nucleic acid strand and hybridized to the target nucleic acid. The example composition further comprises an optional barcode sequence comprised on the circularizable probe or circularizable probe set. The example composition further comprises an optional ligation site comprised within the gap between the two hybridization regions between the circularizable probe or circularizable probe set and the target nucleic acid. The example composition further comprises a second optional ligation site comprised between the gap of the two individual nucleic acid strands comprised within the circularizable circularizable probe set.

In some embodiments, disclosed herein is a composition that comprises a complex containing a target nucleic acid, a circularizable probe or circularizable probe set (e.g., any of the circularizable probes or circularizable probe sets comprised herein), a second nucleic acid strand, and a ligase. In some embodiments, the second nucleic acid strand is crosslinked to the circularizable probe or circularizable probe set. In some embodiments, the circularizable probe or circularizable probe set is hybridized to the target nucleic acid. In some embodiments, the ligase is contacted to a gap in the hybridization region between the circularizable probe or circularizable probe set and the target nucleic acid.

In some embodiments, the composition comprises a splint oligonucleotide. In some embodiments, the splint oligonucleotide is hybridized to the circularizable probe set. In some embodiments, the composition comprises a ligase. In some embodiments, the ligase is contacted to connect a gap in the hybridization region between the circularizable probe set and the target nucleic acid.

In some embodiments, disclosed herein is a composition that comprises a primer or primer oligonucleotide. In some embodiments, the primer or primer oligonucleotide is hybridized to the circularizable probe or circularizable probe set at a primer binding region.

In some embodiments, disclosed herein is a composition that comprises a complex containing a target nucleic acid, a circular nucleic acid template, and a second nucleic acid strand. In some embodiments, the second nucleic acid strand is crosslinked to the circular nucleic acid template. In some embodiments, the circular nucleic acid template is hybridized to the target nucleic acid. An example composition is shown in FIG. 2A.

In some embodiments, disclosed herein is a composition that comprises a complex containing a target nucleic acid, a circular nucleic acid template, a second nucleic acid strand, and a polymerase. In some embodiments, the second nucleic acid strand is crosslinked to the circular nucleic acid template. In some embodiments, the circular nucleic acid template is hybridized to the target nucleic acid. In some embodiments, the polymerase is contacted to the second nucleic acid strand crosslinked to the circular nucleic acid template. An example composition is shown in FIG. 2B.

In some embodiments, disclosed herein is a composition that comprises a complex containing a target nucleic acid, a circular nucleic acid template, a second nucleic acid strand, a polymerase, and an extended nucleic acid priming sequence. In some embodiments, the polymerase disrupts contact between the circular nucleic acid template and the target nucleic acid. FIG. 2B shows example compositions resulting from generation of the extended nucleic acid priming sequence using the circular nucleic acid template as a template.

In some embodiments, the second nucleic acid strand comprises a 3′ block. In some embodiments, the circular nucleic acid template is hybridized to the target nucleic acid. In some embodiments, the target nucleic acid comprises a priming sequence. An example composition is shown in FIG. 3A, wherein the second nucleic acid strand comprising the optional 3′ block is crosslinked to a circular nucleic acid template hybridized to a target nucleic acid.

In some embodiments, the composition comprises an endonuclease. In some embodiments, the second nucleic acid strand comprises a 3′ block.

### VI. Opto-Fluidic Instruments for Analysis of Biological Samples

Provided herein is an instrument having integrated optics and fluidics modules (an “opto-fluidic instrument” or “opto-fluidic system”) for analyzing biological samples (e.g., one or more cells or a tissue sample) as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., one or more probing cycles used for detection and analysis as described in Section III). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (e.g., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

FIG. 5 shows an example workflow of analysis of a biological sample 510 (e.g., cell or tissue sample) using an opto-fluidic instrument 520, according to various embodiments. In various embodiments, the sample 510 comprises a biological sample (e.g., a tissue) that includes molecules such as DNA, RNA, proteins, antibodies, etc. For example, the sample 510 can be a sectioned tissue that is treated to access the RNA thereof for labeling with probes described herein. Ligation of the probes may generate a circular probe which can be enzymatically amplified and bound with detectably labeled probes, which can create bright signal that is convenient to image and has a high signal-to-noise ratio.

In various embodiments, the sample 510 may be placed in the opto-fluidic instrument 520 for analysis and detection of the molecules in the sample 510. In various embodiments, the opto-fluidic instrument 520 can be a system configured to facilitate the experimental conditions conducive for the detection of the target molecules. For example, the opto-fluidic instrument 520 can include a fluidics module 540, an optics module 550, a sample module 560, and an ancillary module 570, and these modules may be operated by a system controller 530 to create the experimental conditions for the probing of the molecules in the sample 510 by selected probes (e.g., circularizable DNA probes), as well as to facilitate the imaging of the probed sample (e.g., by an imaging system of the optics module 550). In various embodiments, the various modules of the opto-fluidic instrument 520 may be separate components in communication with each other, or at least some of them may be integrated together.

In various embodiments, the sample module 560 may be configured to receive the sample 510 into the opto-fluidic instrument 520. For instance, the sample module 560 may include a sample interface module (SIM) that is configured to receive a sample device (e.g., cassette) onto which the sample 510 can be deposited. That is, the sample 510 may be placed in the opto-fluidic instrument 520 by depositing the sample 510 (e.g., the sectioned tissue) on a sample device that is then inserted into the SIM of the sample module 560. In some instances, the sample module 560 may also include an X-Y stage onto which the SIM is mounted. The X-Y stage may be configured to move the SIM mounted thereon (e.g., and as such the sample device containing the sample 510 inserted therein) in perpendicular directions along the two-dimensional (2D) plane of the opto-fluidic instrument 520.

The experimental conditions that are conducive for the detection of the molecules in the sample 510 may depend on the target molecule detection technique that is employed by the opto-fluidic instrument 520. For example, in various embodiments, the opto-fluidic instrument 520 can be a system that is configured to detect molecules in the sample 510 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 510 using reagents such as washing/stripping reagents, hybridizing reagents, etc., and such reagents may be provided by the fluidics module 540.

In various embodiments, the fluidics module 540 may include one or more components that may be used for storing the reagents, as well as for transporting said reagents to and from the sample device containing the sample 510. For example, the fluidics module 540 may include reservoirs configured to store the reagents, as well as a waste container configured for collecting the reagents (e.g., and other waste) after use by the opto-fluidic instrument 520 to analyze and detect the molecules of the sample 510. Further, the fluidics module 540 may also include pumps, tubes, pipettes, etc., that are configured to facilitate the transport of the reagent to the sample device (e.g., and as such the sample 510). For instance, the fluidics module 540 may include pumps (“reagent pumps”) that are configured to pump washing/stripping reagents to the sample device for use in washing/stripping the sample 510 (e.g., as well as other washing functions such as washing an objective lens of the imaging system of the optics module 550).

In various embodiments, the ancillary module 570 can be a cooling system of the opto-fluidic instrument 520, and the cooling system may include a network of coolant-carrying tubes that are configured to transport coolants to various modules of the opto-fluidic instrument 520 for regulating the temperatures thereof. In such cases, the fluidics module 540 may include coolant reservoirs for storing the coolants and pumps (e.g., “coolant pumps”) for generating a pressure differential, thereby forcing the coolants to flow from the reservoirs to the various modules of the opto-fluidic instrument 520 via the coolant-carrying tubes. In some instances, the fluidics module 540 may include returning coolant reservoirs that may be configured to receive and store returning coolants, e.g., heated coolants flowing back into the returning coolant reservoirs after absorbing heat discharged by the various modules of the opto-fluidic instrument 520. In such cases, the fluidics module 540 may also include cooling fans that are configured to force air (e.g., cool and/or ambient air) into the returning coolant reservoirs to cool the heated coolants stored therein. In some instances, the fluidics module 540 may also include cooling fans that are configured to force air directly into a component of the opto-fluidic instrument 520 so as to cool said component. For example, the fluidics module 540 may include cooling fans that are configured to direct cool or ambient air into the system controller 530 to cool the same.

As discussed above, the opto-fluidic instrument 520 may include an optics module 550 which include the various optical components of the opto-fluidic instrument 520, such as but not limited to a camera, an illumination module (e.g., LEDs), an objective lens, and/or the like. The optics module 550 may include a fluorescence imaging system that is configured to image the fluorescence emitted by the probes (e.g., oligonucleotides) in the sample 510 after the probes are excited by light from the illumination module of the optics module 550.

In some instances, the optics module 550 may also include an optical frame onto which the camera, the illumination module, and/or the X-Y stage of the sample module 560 may be mounted.

In various embodiments, the system controller 530 may be configured to control the operations of the opto-fluidic instrument 520 (e.g., and the operations of one or more modules thereof). In some instances, the system controller 530 may take various forms, including a processor, a single computer (or computer system), or multiple computers in communication with each other. In various embodiments, the system controller 530 may be communicatively coupled with data storage, set of input devices, display system, or a combination thereof. In some cases, some or all of these components may be considered to be part of or otherwise integrated with the system controller 530, may be separate components in communication with each other, or may be integrated together. In other examples, the system controller 530 can be, or may be in communication with, a cloud computing platform.

In various embodiments, the opto-fluidic instrument 520 may analyze the sample 510 and may generate the output 590 that includes indications of the presence of the target molecules in the sample 510. For instance, with respect to the example embodiment discussed above where the opto-fluidic instrument 520 employs a hybridization technique for detecting molecules, the opto-fluidic instrument 520 may cause the sample 510 to undergo successive rounds of detectably labeled probe hybridization (e.g., using two or more sets of fluorescent probes, where each set of fluorescent probes is excited by a different color channel) and be imaged to detect target molecules in the probed sample 510. In such cases, the output 590 may include optical signatures (e.g., a codeword) specific to each gene, which allow the identification of the target molecules.

### VII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some embodiments, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or 1), ii), etc. does not by itself connote any priority, precedence, or order of steps in the claims. Similarly, the use of these terms in the specification does not by itself connote any required priority, precedence, or order.

### Examples

The following examples are included for illustrative purposes only and are not intended to limit the scope of the present disclosure.

### Example 1: Method for In Situ Detection of a Target Nucleic Acid Utilizing a Circular Nucleic Acid Template Crosslinked to a Second Nucleic Acid Strand Prepared In Vitro

This example describes a method for in vitro preparation of a circular nucleic acid template and detection of a target nucleic acid within a biological sample using the template.

For in situ detection of a target nucleic acid that involves amplification of probes that hybridizes to the target nucleic acid (e.g., rolling circle amplification) and detection of the amplification product, challenges with generating uniform amplification products can result from unsynchronized initiation and/or progression of amplification between individual molecules. A potential solution to this problem is to synchronize amplification in situ. This example discloses one potential solution utilizing circular nucleic acid templates crosslinked to a second nucleic acid strand.

As shown in FIG. 1A, an example circularizable probe comprises both target-complementary arms (e.g. regions on the 3′ and 5′ ends of the probe that hybridize to the target nucleic acid) and a sequence that hybridizes to a second nucleic acid strand (e.g., a primer). The example circularizable probe also comprises a detectable sequence (e.g., a barcode). The example second nucleic acid strand comprises one or more bases comprising moieties capable of crosslinking to another molecule (e.g., 3-cyanovinylcarbazole; ^{CNV}K). The probe and the second strand are contacted in vitro (e.g., within a tube) and subsequently crosslinked (e.g., by hybridization between the probe and primer and exposure to UV light for 1-30 seconds).

The probe crosslinked to the second nucleic acid strand is then contacted with a biological sample to hybridize to a target nucleic acid in situ, as shown in FIG. 1B. In this example, the biological sample undergoes one or more stringent washing steps to remove any probes that have not hybridized to their respective target nucleic acid. After hybridization, probes are efficiently ligated (e.g., through template-mediated ligation) to form a circular nucleic acid template comprising a crosslinked second nucleic acid strand (FIG. 2A).

The circular nucleic acid template is used as a template to generate an extended nucleic acid priming sequence. As shown in FIG. 2B, the biological sample is contacted with a polymerase (e.g., Phi29) that may bind to the second nucleic acid strand crosslinked to the ligated template. After binding, the polymerase generates an extended nucleic acid priming sequence using the circular nucleic acid template as a template until it reaches the crosslink (FIG. 2B). Once at the crosslink position, the polymerase stalls. Optionally, the sample is contacted with a second polymerase. Without being bound by theory, the stalling and optional contacting with a second polymerase allows improved synchronization of RCA across a plurality of RCA reactions in the biological sample.

As shown in FIG. 2C, the polymerase continues to amplify following reversal of the crosslink (e.g., exposure of the biological sample to UV light at 312 nm). Reversal of the crosslink allows for amplification of the circular nucleic acid template (e.g., rolling circle amplification; RCA) and subsequent detection (e.g., sequential binding of detectably labeled probes, sequencing by synthesis, sequencing-by-binding, or sequencing-by-avidity). Without being bound by theory, through both stalling of the polymerase, optional contacting of the biological sample with new polymerase, and reversal of crosslinking, this method provides several points wherein amplification may be synchronized across the entire biological sample. In this way, crosslinking between the circular nucleic acid template and the second nucleic acid strand may result in better uniformity and detection of signal.

### Example 2: Method for In Situ Detection of a Target Nucleic Acid Utilizing a Circular Nucleic Acid Template Crosslinked to a Second Nucleic Acid Strand Comprising a 3′ Block

This example describes a method for detection of a target nucleic acid within a biological sample utilizing a circular nucleic acid template crosslinked to a second nucleic acid strand, wherein the second nucleic acid strand comprises a 3′ block.

As shown in FIG. 3A, an example circular nucleic acid template comprises both target-complementary arms (e.g. regions on the 3′ and 5′ ends of the probe that hybridize to the target nucleic acid) and a sequence that hybridizes to a second nucleic acid strand (e.g., a primer). The template also comprises an optional detectable sequence (e.g., a barcode). The example second nucleic acid strand comprises one or more bases capable of crosslinking to another molecule (e.g., 3-cyanovinylcarbazole; ^{CNV}K). The second nucleic acid strand also comprises an optional 3′ block (e.g., an irreversible terminating group on the 3′ end).

As outlined in Example 1, the circular nucleic acid template is formed upon hybridization of a circularizable probe or circularizable probe set to the target nucleic acid and ligation (e.g., template-mediated ligation). In some cases, upon formation of the circular nucleic acid template (e.g., a circular DNA template), digestion and/or cleavage by endonucleases (e.g., RNaseH) allows the target nucleic acid (e.g., a target RNA) hybridized to the circular nucleic acid to be used as a priming sequence by the polymerase for extension. A first round of amplification by the polymerases will generate an extended nucleic acid priming sequence. Polymerases may also hybridize to the second nucleic acid strand crosslinked to the circular nucleic acid template. Without being bound by theory, presence of the 3′ block on the second nucleic acid strand prevents generation of a second extended nucleic acid priming sequence by the polymerase. After the polymerase stalls at the crosslink position, extension resumes upon reversal of the crosslink and subsequent detection is performed as described in Example 1.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method, comprising:
(a) extending a nucleic acid priming sequence hybridized to a circular nucleic acid template using a polymerase to generate an extended priming sequence, wherein a first nucleic acid strand comprises the circular nucleic acid template, and the circular nucleic acid template is crosslinked to a second nucleic acid strand;
(b) de-crosslinking the circular nucleic acid template from the second nucleic acid strand; and
(c) extending the extended priming sequence to generate a rolling circle amplification product (RCP).

2. (canceled)

3. A method, comprising:
(a) extending a target nucleic acid hybridized to a circular nucleic acid template using a polymerase to generate an extended sequence, wherein a first nucleic acid strand comprises the circular nucleic acid template, and the circular nucleic acid template is crosslinked to a second nucleic acid strand separate from the target nucleic acid;
(b) de-crosslinking the circular nucleic acid template from the second nucleic acid strand; and
(c) extending the extended sequence to generate a rolling circle amplification product (RCP).

4. (canceled)

5. The method of claim 1, wherein the polymerase is a Phi29 DNA polymerase or a Bst DNA polymerase.

6. The method of claim 5, wherein the method comprises providing a second polymerase after de-crosslinking the circular nucleic acid template from the second nucleic acid strand, wherein the second polymerase is a Phi29 DNA polymerase or a Bst DNA polymerase.

7. The method of claim 1, wherein the method is performed in a biological sample, and
wherein the method comprises contacting the biological sample with the second nucleic acid strand and:
(i) contacting the biological sample with the circular nucleic acid template, wherein the circular nucleic acid template hybridizes to a target nucleic acid in the biological sample, or
(ii) contacting the biological sample with a circularizable probe or circularizable probe set that hybridizes to a target nucleic acid in the biological sample, and ligating the circularizable probe or the circularizable probe set to generate the circular nucleic acid template in the biological sample.

8-10. (canceled)

11. The method of claim 7, wherein the second nucleic acid strand is crosslinked to the circular nucleic acid template or the circularizable probe or the circularizable probe set before contacting the biological sample, wherein the second nucleic acid strand is crosslinked to the circular nucleic acid template or the circularizable probe or the circularizable probe set by irradiating a mixture comprising the second nucleic acid strand and the circular nucleic acid template, the circularizable probe, or the circularizable probe set with UV light prior to contacting the biological sample.

12-13. (canceled)

14. The method of claim 11, wherein the mixture is irradiated using a 350-400 nm wavelength of light.

15-18. (canceled)

19. The method of claim 1, wherein the second nucleic acid strand comprises the nucleic acid priming sequence.

20. The method of claim 1, wherein the second nucleic acid strand does not comprise the nucleic acid priming sequence.

21-24. (canceled)

25. The method of claim 7, wherein the target nucleic acid is RNA.

26-28. (canceled)

29. The method of claim 7, wherein the circular nucleic acid template, the circularizable probe, or the circularizable probe set comprises a hybridization region that hybridizes to the target nucleic acid, and wherein the hybridization region comprises a crosslinkable moiety.

30. (canceled)

31. The method of claim 1, wherein the second nucleic acid strand comprises a crosslinkable moiety.

32. The method of claim 31, wherein the crosslinkable moiety of the second nucleic acid strand is at the 5′ end of the second nucleic acid strand or within 1, 2, 3, 4, or more nucleotides from the 5′ end of the second nucleic acid strand.

33-41. (canceled)

42. The method of claim 1, wherein de-crosslinking the circular nucleic acid template from the second nucleic acid strand comprises irradiating the circular nucleic acid template crosslinked to the second nucleic acid strand.

43-45. (canceled)

46. The method of claim 1, wherein the method comprises detecting the RCP, and wherein the RCP is detected at a location in the biological sample or a matrix embedding the biological sample.

47-52. (canceled)

53. The method of claim 3, wherein the target nucleic acid is:
(a) a cellular nucleic acid analyte or a product thereof;
(b) associated with a non-nucleic acid analyte, wherein the target nucleic acid is an oligonucleotide reporter in a labeling agent that binds to the non-nucleic acid analyte; or
(c) a probe or probe set associated with a nucleic acid analyte or product thereof in the biological sample.

54-57. (canceled)

58. The method of claim 7, wherein the biological sample is a cell or tissue sample.

59. (canceled)

60. The method of claim 7, wherein the biological sample is permeabilized.

61-64. (canceled)

65. A kit, comprising:
a circular probe or a circularizable probe or a circularizable probe set, wherein a first nucleic acid strand comprises the circular probe or the circularizable probe, or a set of first nucleic acid strands comprises the circularizable probe set, and the circularizable probe or the circularizable probe set is capable of being circularized upon hybridization to a target nucleic acid, and
a second nucleic acid strand comprising a sequence complementary to a sequence of the circular probe, the circularizable probe, or the circularizable probe set, wherein the sequence complementary to the sequence of the circular probe, the circularizable probe, or the circularizable probe set comprises a crosslinkable moiety.

66-70. (canceled)

71. The method of claim 1, wherein the circular nucleic acid template is generated by ligating a padlock probe prior to (a).
